(19) United States
(12) Patent Application Publication
(10) Pub. No.: US 2025/0027863 A1
(43) Pub. Date: Jan. 23, 2025

(54) METHOD OF CHARACTERIZATION OF RELEASE OF POORLY SOLUBLE MATERIALS AND UPTAKE IN COMPLEX LIQUIDS

(71) Applicants: Zennova LLC, Princeton, NJ (US); Physical Pharmaceutica LLC, Orangeburg, NY (US)

(72) Inventors: Robert A. Bellantone, Yonkers, NY (US); Hengsheng Feng, Moorestown, NJ (US)

(73) Assignees: Zennova LLC, Princeton, NJ (US); Physical Pharmaceutica LLC, Orangeburg, NY (US)

(21) Appl. No.: 18/706,918

(22) PCT Filed: Feb. 16, 2022
(86) PCT No.: PCT/US2022/016610

§ 371 Date: May 2, 2024

Related U.S. Application Data
(60) Provisional application No. 63/360,840, filed on Nov. 3, 2021.

(51) Int. Cl. G01N 13/00; G01N 33/15

(52) U.S. Cl. CPC G01N 13/00; G01N 33/15; G01N 2013/003

(57) ABSTRACT

An improved method of measuring transfer of agents from solutions, emulsions, otherwise dispersed structures between two or more media that are separated by suitable membranes, characterized by the loss of the agent(s) from the medium in which it is contained and/or the collection by the other medium. Preferred applications include characterizing the rates and extents at which transfer processes occur, such as from emulsions, microemulsions, and nanoemulsions, suspensions of particles, or complexes; determining the rate of dissolution of a drug from a suspension; determining the rate of a reaction or complexation or freeing of the drug from a complexed to dissolved form; determining the rate of a chemical reaction of a drug in a medium; determining the solubility of a drug in a homogeneous or heterogeneous liquid medium. The invention includes newly created apparatus, and produces remarkably fast and precise results, permitting measurements not previously possible to make.

This application claims the priority of U.S. Provisional application No. 63/360,840, filed on Nov. 3, 2021. The content of the application is incorporated herein by reference in its entirety.

### BACKGROUND OF THE INVENTION

### Field of the invention

One of the most important pharmaceutical properties of a pharmaceutical formulation is its ability to transfer drug molecules from forms or states from which absorption into physiological tissues is poor into forms more readily available for absorption. As used herein, the term “absorption” refers to the ability of a drug to enter or partition into a biological membrane (such as stomach or intestinal wall, skin, the cornea, cell walls, etc.) and perhaps permeating the membrane by diffusion or carrier-mediated transport. Most often, such absorption requires the drug molecules to be dissolved in a medium that is in contact with the biological membranes, since only individual drug molecules can partition into a membrane and permeate it by diffusion. It is generally accepted that dispersed structures such as emulsion globules, micelles, undissolved particles, polymers and proteins are too large to pass through such membranes in this manner. Thus, drug molecules that are contained in such dispersed structures, or bound to polymers and other large carrier agents, are unable to enter and permeate biological membranes. (Oral absorption can for some drugs if they are incorporated into fat globules that are digested. However, this is not a preferred means of drug delivery for the vast majority of drugs and is not discussed further herein.)

Some important examples include liquid topical ophthalmic formulations, including dispersions such as emulsions and suspensions. These are formulations in which emulsion globules, micelles or undissolved particles containing a drug are dispersed in water or an aqueous phase (herein referred to as the aqueous or continuous phase). Typically, these formulations are employed for drugs that are poorly soluble in water and most or nearly all of the drug in the formulation is not dissolved in the aqueous phase. Further, these formulations are typically about 90 percent or more water, so the aqueous phase dominates the area of contact between the formulation and the target biological membranes. As a result, the drug that is dissolved in the aqueous phase dominates the absorption into tissues, while most of the drug in the formulation (at least initially on administration) is not present in the aqueous phase. Due to absorption into tissues or dilution with physiological fluids, the drug will redistribute from the “unabsorbable” form or state (for instance, undissolved particles, drug partitioned into globules, drug molecules complexed with proteins or polymers) into the absorbable form where it is dissolved and free in the aqueous phase. (It not necessary for the continuous phase to be aqueous. For instance, suspensions of undissolved particles in oil vehicles are known in the art. However, formulations comprising an aqueous continuous phase are of particular pharmaceutical interest and used as the examples in the discussions herein.)

Other examples include “drug-carrier” formulations, in which a drug is bound to or complexed with a carrier such as a polymer or protein. In these formulations, the drug is present in both bound and free forms, and the change in the distribution with time is useful for evaluating and comparing formulations.

While ophthalmic dispersions and drug-carrier complexes represent two categories of examples, this should not be interpreted as limiting the scope of the field and could include other examples of multiple processes occurring during the release and redistribution of a drug or other agent.

In what follows, the term “release” will be used in the context of a “release test” (in vitro or in vivo) and refers to the drug that is lost from or gained by a test sample, or gained by a receiving test medium, and the term “IVRT” is an acronym for “in vitro release test.” Also, “release test” will refer to a test in which the “drug release” is a measured quantity or fraction of a drug that is gained or lost by a medium being sampled. The term “release profile” will refer to said amount or fraction gained or lost vs. time or some other parameter.

As used herein, the term “redistribution” refers to the transfer of drug molecules to the dissolved form (typically in the continuous phase of the dispersion) from undissolved dispersed particles by dissolution, migration of dissolved drug molecules out of globules, the freeing of drug molecules from binding or complexation with proteins, polymers or other agents, etc. Redistribution can also describe transfer in the other direction, in which drug molecules dissolved in a continuous phase leave that state by such processes as precipitation, distribution into globule phases, or binding.

It is noted that, in the literature and the art, the term “release” has been used in more than one context for different applications, including when a molecule changes from a complexed or undissolved state to its free or dissolved form. As discussed above, these will be termed herein as redistribution rather than release. The processes in which a drug that is complexed with or bound to a carrier is transferred to the free unbound form will be referred to herein as “freeing” or described as the transfer or redistribution from the bound or complexed form to the free form.

It is of interest in the pharmaceutical sciences to measure or characterize the ability of drugs in formulations to redistribute from an unabsorbable form into a continuous (usually aqueous) medium from which absorption occurs. Information regarding the initial distribution and rate and extent of such redistribution in controlled circumstances can be used to characterize the properties of drug materials and formulations and help to compare the performance of formulations in terms of how they make a drug available to for absorption. This is done experimentally using release tests for which, at least in principle, the rate or extent of drug release depends on the drug initial distribution and redistribution that occurs during the test. Typically, IVRTs are preferred over in vivo release tests because they are usually more convenient, less expensive, easier to perform and control, and if expertly done using the proper equipment, quickly produce more accurate and detailed results than otherwise possible.

For the purpose of evaluating or comparing complex pharmaceutical formulations, certain general considerations are relevant.

For ophthalmic dispersion formulations, the distribution of the drug is a critical factor affecting the drug release or delivery to the body. This is because not all forms of a drug are available to the physiological tissues to the same degree or are released at the same rate. For instance, it is thought that a drug in an ophthalmic emulsion is delivered to the ocular tissues essentially exclusively from the aqueous phase. The drug that is dissolved in the oil and surfactant phases cannot pass directly to the tissues but, instead, redistributes or transfers out of the oil and surfactant phases into the aqueous phase, from which it then can be delivered to the ocular tissues. In an IVRT, this redistribution is triggered because of the reduced drug content in the aqueous phase due to drug loss or release to a receiver medium. Further, an IVRT is physiologically relevant only if data that accurately reflect the release and resulting distribution are obtained over short enough timeframes to reflect the short residence time of the formulation in the ocular region—data that are insensitive to changes that occur in the physiological timeframe are useless for comparing in vivo performance in the ocular region.

In addition, it is known that a lipophilic drug can transfer slowly from an oil to aqueous phase so the redistribution from the oil to aqueous phase is much slower than the release from the aqueous phase to a tissue. On the other hand, it is also well-known that a transfer of a drug from dispersed micelles into an aqueous phase is a rapid process. (These cases are relevant since ophthalmic emulsions are primarily used to formulate lipophilic drugs and may also contain micelles.) Thus, the IVRT must also be able to release the drug from the formulation into the receiver at a fast enough rate to allow the data to distinguish between the faster vs. slower processes. In general, this requires that the medium being sampled (the formulation or the receiver) should occupy a small volume, and the ratio of the area in which the exchange occurs between the formulation and receiver, referred to as the area-to-volume ratio, is sufficiently large to allow significant changes in the sample concentration due to exchange with the receiver to occur over times that are not so long as to be physiologically irrelevant. This is because the rate of mass transfer between a formulation and receiver scales with the exchange area while the change in concentration in the medium being sampled (formulation or receiver) scales as the inverse of its volume being sampled.

Most of the considerations that apply to ophthalmic emulsions also apply to ophthalmic suspensions, with the difference being that the drug release from the aqueous phase induces a dissolution of the undissolved drug from particles dispersed in the suspension. Analogously, for a drug-carrier formulation, the drug that is bound or complexed, the response process that is induced by loss of the drug from the aqueous medium is a redistribution from the complexed to free form of the drug.

In the above examples, an IVRT should provide a means of causing and measuring a drug release that triggers drug redistribution in the formulation as a response to replace the drug lost from the aqueous phase. The release represents a loss of the dissolved drug from the aqueous phase of the formulation to a receiving medium. The redistribution process to replace the drug lost to release from the aqueous phase would depend on the formulation, such as drug molecules transferring from globules and perhaps micelles in an emulsion, dissolving from undissolved particles in a suspension, or leaving the complexed state in a drug-carrier formulation. Similar considerations would apply to drugs that are solubilized in micellar systems.

Without loss of generality, the release of a drug in an IVRT can be measured by sampling the drug formulation and determining the amount of the drug that was lost due to release, or by sampling the drug receiver and determining the drug released from the formulation from the accumulation of the drug in the receiver. However, there are certain limitations. For instance, if the volume of the formulation is much larger than the receiver volume in the IVRT, the drug released from the formulation would be minor compared to the total amount of the drug in the formulation, so any resulting fractional change in the total drug concentration (used to determine the drug release) would be very small. Similarly, if the receiver volume is large, the amount of the drug that it accumulates would also result in a small concentration change. In either case, the formulation and receiver volumes must be chosen carefully to ensure the data are properly sensitive.

For some end-applications, designing a suitable IVRT is a challenging problem, especially if one of the goals is to assess factors relevant to their performance in vivo (i.e., a “biorelevant” IVRT). For instance, release testing of ophthalmic dispersion formulations such as emulsions and suspensions must be performed rapidly due to the short residence time that these products have in the ocular region. Most of a drop instilled into the eye is cleared from the ocular region in seconds to a few minutes due to runoff, blinking, and subsequent tearing. Thus, the time for drug delivery to the ocular surface and redistribution occurring in the formulation during its residence in the ocular region is short. In addition, any in vivo redistribution from an administered drop of an ophthalmic dispersion is into a receiver of low volume that is presumably similar to the volume of a tear layer plus tears that are produced. For this case, data obtained from a biorelevant IVRT would be obtained in this timeframe and, also, show differences between formulations with physicochemical differences.

Another end-application of interest would be for injectable formulations in which the drug is complexed with carriers such as proteins or polymers. Since the drug molecules must be free and dissolved (unbound or not complexed) before bringing about a pharmacological effect, factors relevant to the in vivo drug redistribution from the complexed to dissolved free form, and IVRTs that can characterize the rates and extents of such redistributions, are of great potential utility in designing and comparing such injectable formulations.

Ophthalmic dispersions and drug-carrier complexes are referred to as “complex” formulations or dosage forms. As used herein when referring to a formulation or dosage form, the term “complex” formulation or dosage form refers to a medium comprising multiple phases with different properties into which a drug is distributed. This use is different from using the term “complex” to describe a bound state of a drug and a carrier such as a polymer or protein. The meaning of the term “complex” should be clear from its context to one of skill in the art, but it will be specified herein if there is any apparent ambiguity.

An ophthalmic emulsion comprises globules of oil and/or surfactant, which are dispersed in an aqueous continuous phase that typically accounts for about 90% or more of the formulation by weight. In these emulsions, a drug is typically completely dissolved, but the dissolved drug is distributed into the oil, water and (likely) surfactant phases. For drugs with poor aqueous solubility, which is a primary application of ophthalmic emulsions, only a small fraction of the total drug in the emulsion is dissolved in the aqueous phase. An ophthalmic suspension comprises a dispersion of undissolved drug particles in an aqueous continuous phase in which some of the drug is also dissolved, most typically more than about 90% of the total drug content being undissolved. It is thought that the drug redistribution that occurs on administration of these formulations is affected by the distribution of the drug in these phases, which depends on the phase properties and the distribution of other excipients in formulations, which may be in ways that are not completely characterized or understood.

For ophthalmic formulations such as emulsions and suspensions, the ocular physiology and in vivo conditions dictate attributes that are desirable or even necessary for in vitro tests to be biorelevant. For instance, due to the short residence time of a drop in the ocular region when administered, it is important for an IVRT to characterize drug release from within the first minute onward to allow inferences regarding in vivo processes. Also, due to the small volume of ocular fluids present in vivo, such as tears, a relevant in vitro test should avoid large dilutions or other factors affecting the physical properties of a formulation during testing.

It can also be important to determine the initial distribution of a drug in a formulation (before the administration) and the redistribution that occurs when the system equilibrium is disturbed. For instance, the drug delivery properties of an ophthalmic emulsion can critically depend on the initial fraction of a drug in the aqueous phase vs. the oil globules. Further, release of the drug from the aqueous phase will induce redistribution of the drug out of the globules and into the aqueous phase. In general, the timeframes characterizing the release from the aqueous phase vs. redistribution from the oil globules are different. Knowledge of the rates of release versus induced redistribution rates can be useful when determining how a formulation works to deliver the drug, and in comparing multiple formulations for similarity. This is well known to the pharmaceutical industry. For instance, the U.S. Food and Drug Administration (FDA) requests IVRTs for generic drug applications for a number of ophthalmic products. (See, for example, Draft Guidance on Cyclosporine, October 2016; Draft Guidance on Difluprednate, February 2017; Draft Guidance on Prednisolone Acetate, May 2019.)

In addition, test data can be analyzed using appropriate models to relate release and redistribution processes and the initial drug distribution in various phases of a formulation, etc. that occur during a release test. For instance, appropriate models would make it is possible to obtain dissolution data for suspended particles as a function of time during an IVRT from the release data. However, before such analyses can be performed, data must be collected that can distinguish between fast and slow timeframes.

While the above discussion primarily focuses on ophthalmic products, it should not be inferred that the instant invention is limited to these formulations. The concepts also apply to other topical, oral, and injectable formulations comprising liposomes, oily suspensions, drug-carrier complexes, and numerous others, as would be apparent to one of skill in the art.

An example of another end-application of interest would be developing IVRTs for drug-carrier formulations, such as an injectable formulation comprising a drug that is bound to or complexed with a protein, polymer, antibody, or other carrier. Since the drug molecules must be free and dissolved (unbound or not complexed) before bringing about a pharmacological effect, factors relevant to the in vivo drug redistribution from the complexed to dissolved free form, and IVRTs that can characterize the rates and extents of such redistributions, are of great potential utility in designing and comparing such injectable formulations. For instance, the rates and extents of drug-carrier binding and freeing (“unbinding”) under different chemical and physical circumstances can be helpful for comparing drug-carrier injectable formulations for sameness, or for developing manufacturing processes.

Obtaining such in vitro characterization of the drug release from a formulation can provide critical information for developing an ophthalmic formulation or assessing formulations for purposes of quality control, in vitro bioavailability comparisons, or potential drug modeling such as physiologically based pharmacokinetics (PBPK). However, no prior technology has satisfied these requirements. This is evidenced by a lack of published data on the rates and extents of processes occurring in ophthalmic emulsions during drug release. It is further evidenced by the lack of acceptance of any release or dissolution tests by regulatory agencies, such as the FDA, for the purposes of establishing bioequivalence between a brand and generic product.

Importantly, the in vivo performance of above-mentioned complex formulations derives not only from their chemistry and composition, but also from the physical structure (for instance, emulsion vs. solution), which governs the release, distribution and redistribution properties and behavior of a formulation. The structure is also referred to as the “arrangement of matter” or “Q3” by the FDA. (See, for instance, numerous product-specific guidances or PSGs, such as the avoe-referenced Draft Guidance on Cyclosporine and Draft Guidance on Difluprednate.)

IVRTs that employ large receiver volumes may not be biorelevant and, thus, not indicative of how a drug is released in vivo. For instance, sufficient dilution of a formulation can disrupt its physical structure and properties in a manner that would not occur in vivo, where the Q3 design was intended to apply. In those cases, the IVRT may be more reflective of the disruption of the formulation structure than the release and redistribution processes that occur under physiologically relevant conditions, which can result in the IVRT being unable to distinguish between fast vs. slow processes that occur on administration in vivo. Similarly, a formulation dilution can also be inappropriate for drug-carrier formulations because it can cause a redistribution of the drug from the complexed to a free dissolved form that is much more rapid that would occur in vivo.

In addition, when in vivo release or dissolution occurs into limited receiver volumes (e.g., tears), the drug release can create an increased drug concentration in those fluids that affects the subsequent drug release from and redistribution within the formulation. This is especially true for drugs with poor aqueous solubility, as are typically used in ophthalmic emulsions and suspensions. IVRTs that rely on release or dissolution into large receiver volumes create a condition in which the concentration of the drug accumulated in the receiver stays very low (approximated as nearly a zero concentration, referred to as “sink conditions”), so the IVRT is not influenced by an accumulating drug concentration in the receiver liquids and does not accurately reflect the in vivo dissolution conditions.

Aside from dilution issues, the IVRT setup must also avoid slow, rate-limiting steps that prevent detecting fast vs. slow processes. For instance, it is required for ophthalmic emulsions and suspensions that the sampling start at early enough times and be done often enough so faster processes can progress significantly during the sampling intervals while slower processes progress to a far smaller degree. As an example, if the release occurs in an IVRT by a drug crossing a membrane, the membrane must not slow the drug passage to the point where samples take too long to collect, otherwise the detection of fast processes may be delayed so the data cannot adequately separate or distinguish the effects of fast processes vs. slower processes. This can also occur when the volume being sampled is too large. If the formulation volume is too large, then the fractional rate of change in mass within the sample due to release may be too small to be sensitively detected or characterized in the data. Similarly, if the receiver is too large, the fractional rate of change in the drug concentration due to accumulation may also be too small to be sensitively detected or characterized.

From the above discussion, it is apparent that there is a need for improved in vitro release tests or procedures for certain formulations, potentially including commercially available ophthalmic emulsions and suspensions, and drug-carrier complexes. Ideally, an IVRT should track multiple timeframes in one test and distinguish between fast and slow processes. This should facilitate evaluating and comparing drug formulations, and if done in a biorelevant context, provide important information to predict the behavior of the formulations when administered in vivo.

### SUMMARY OF THE INVENTION

The invention comprises a new method for determining rates of transferring a drug or other agent from or between phases or regions in a liquid-based medium either inside or outside of one or more exchange chambers, and for characterizing different rates of transfer between different phases or regions. This method will be referred to as MCRR (Multi-Chamber Release and Response).

The invention provides a method for causing an accurately controlled or characterized change in the amount of a drug or other agent in one phase of a composition that acts as a perturbation to induce processes in response, in which the drug or agent transfers or redistributes among the phases of the composition. The perturbation is the mass exchange of a drug or agent between two media, a flow medium while in an exchange chamber and an external medium in which the exchange chamber is immersed, that are separated by a membrane (exchange chamber membrane) that comprises an MCRR probe. In particular, the instant invention provides a method for measuring the transferred mass of drug or other agent as a function of the time spent in the exchange chamber(s) (the exposure time) from the redistribution processes that occur in response to the perturbation as different functions of time.

In essence, the invention provides a method for accurately determining the rate and extent of exchange of a diffusible agent between a flow medium and external medium, comprising:
a) providing one or more exchange chambers comprising a section of relatively highly permeable membrane relative to any materials to which the membrane is attached for support and positioned between an inlet to a reservoir acting as a source of flow medium and an outlet back to said reservoir source, and through which membrane the diffusible agent is to be transferred;
b) putting each exchange chamber in contact with said external medium by immersing it sufficiently in the external medium so exchange of said diffusible agent can occur between the flow medium and external medium;
c) perfusing a known volume of a flow medium from a source through the relatively highly permeable section of the exchange chamber at a known flow rate;
d) withdrawing a sample of known volume of said flow medium from said reservoir;
e) assaying the sample to determine the concentration and amount of said diffusible agent in the flow medium at the time the sample was taken;
f) optionally, continuing the flow of the flow medium and repeating steps (c)-(e) for a selected number of samples and sampling times.

Steps (a)-(f) allow plotting the concentration and mass of the agent in each sample as a function of time. Further, if the initial concentration and mass content of the agent is determined initially, the concentration gain or loss mass can be determined as a function of time, allowing further data analysis and modeling to be performed.

The procedure outlined in steps (a) through (f) allows for variations, but such variations do not change the instant invention and fall within its scope. For instance, there can be multiple exchange chambers that can have the same or different properties and geometries, arranged in series with a single pump and flow rate or in parallel using multiple pumps and flow rates. Also, while each exchange chamber is immersed in one external medium, more than one exchange chamber can be immersed in the same external medium vessel, or each exchange chamber can be immersed in a separate external medium with its own composition, volume, temperature, etc. Additionally, different flow rates can be utilized for different exchange chambers, or the flow through any given exchange chamber can be varied over time by changing the flow rate and/or the direction of the flow.

In a preferred embodiment, steps (a) through (f) are repeated multiple times and the collected volumes are combined to make the MCRR sample used for assay, and there is no resting time at the end of step (c) or step (d). In another preferred embodiment, the sample withdrawn from the reservoir is replaced by a liquid medium in a volume that may be the same or different from that of the sample, and with a liquid medium of composition that may be the same or different from the composition of the withdrawn sample.

In still another preferred embodiment, the flow medium is exchanged between two reservoirs by successive pulling and pushing, with the total pull volume Vᵣ is the same as the total push volume V_{f}, and both are approximately the same as the exchange chamber volume V_{W} for each cycle, described as:
a) providing one or more exchange chambers comprising a section of relatively highly permeable membrane relative to any materials to which the membrane is attached for support and positioned between a segment of tubing partially immersed in one reservoir and another segment of tubing partially immersed in another reservoir, and through which membrane the diffusible agent is to be transferred;
b) putting each exchange chamber in contact with said external medium by immersing it sufficiently in the external medium so exchange of said diffusible agent can occur between the flow medium and external medium;
c) perfusing a known volume of a flow medium from the first reservoir, through the relatively highly permeable section of the exchange chamber, and into the second reservoir at a known flow rate;
d) optionally, reversing the direction of the flow and perfusing a known volume of a flow medium from the second reservoir, through the relatively highly permeable section of the exchange chamber, and into the first reservoir at a known flow rate;
e) withdrawing a sample of known volume of said flow medium from said second reservoir;
f) assaying the sample to determine the concentration and amount of said diffusible agent in the flow medium at the time the sample was taken;
g) optionally, continuing the flow of the flow medium and repeating steps (c)-(e) for a selected number of samples and sampling times.

It may be necessary to know the exchange chamber volume V_{X} and permeability P of the exchange chamber membrane to facilitate data analysis for certain applications. These can be determined for cylindrical exchange chambers using previously disclosed methods [Bellantone, 2012] and one of skill in the art would be able to use the procedures disclosed to characterize other exchange chamber geometries.

Beyond flow patterns used in the MCRR, there are two other general types of embodiments, one in which the net migration of agent is from the flow medium to the external medium (the flow medium is the donor), and the other in which the net migration of agent is from the external medium to the flow medium (the flow medium is the receiver). As examples, but without implied limitation, a flow medium acting as a donor may comprise a solution, emulsion, or suspension for testing. A flow medium acting as the receiver may comprise a solution that might include a dissolved polymer, binding agents, surfactants, or may be dispersed systems such as emulsions and suspensions.

In a preferred embodiment, exchange chambers comprise a permeable cylindrical membrane that is connected to cylindrical inlet and outlet tubing, but the invention is not limited to exchange chambers with that geometry. Other probe geometries and types can also be used within the scope of the instant invention. (For example, CMA 7 Microdialysis Probes and the like, Harvard Apparatus, Holliston, MA).

FIG. 1 shows a schematic diagram of an MCRR probe in a preferred setup, which employs one reservoir, one exchange chamber immersed in an external medium, and one pump. The probe consists of a permeable to highly permeable segment of tubing (the exchange chamber) that is connected to two segments of impermeable tubing, with one end connected to a needle/syringe and serving as the inlet and the other connected to the exit end of the exchange chamber and serving as outlet tubing from which samples are collected.

Typically, each exchange chamber volume ranges from about 1-10 μL, but it can be smaller or larger in practice. Most practically, the exchange chamber volume is smaller than the volume of the impermeable tubing in the MCRR probe, but this is not required to practice the invention. Also, increasing the ratio of the exchange chamber's surface area to its volume (the area-to-volume ratio) can increase the time sensitivity, e.g., the ability to detect release or uptake over shorter sampling intervals and more frequently. This is because 1) the rate of exchange between the flow medium and external medium increases with increasing area, and 2) for any change in the amount of a drug in the flow medium, a smaller volume will result in a larger concentration change. Importantly, the exchange between the flow medium and external medium in any short time interval (such as the time a volume element of the flow medium is in the exchange chamber) involves the release mechanism because only the drug that is dissolved in the aqueous or continuous phase of the flow medium can diffuse across the exchange chamber membrane. Release rates are indirectly affected by redistribution, which is induced as a response to release and often involves a replacement of the released drug that occurs at a rate that is slower than the rate of loss due to release. Thus, increasing the exchange chamber area-to-volume ratio improves the ability of the MCRR test to collect data frequently enough to distinguish between relatively rapid and slow processes (such as release vs. redistribution) that occur during a release test.

In its broadest sense, the instant invention relates to immersing an exchange chamber in an external medium, then causing a flow medium fluid (typically a liquid formulation) to be withdrawn from a reservoir vessel, flowing said flow medium through the exchange chamber, and returning it to the reservoir vessel. While passing through the exchange chamber, drugs or other agents can be lost from the flow medium in the exchange chamber to the external medium (acting as a receiver) or accumulated in the flow medium in the exchange chamber from the external medium (acting as a donor) by diffusion or other transport across the permeable exchange chamber membrane. Typically, the flow medium is passed through the MCRR probe at a constant flow rate Q and in one direction, being withdrawn from a reservoir, passed through the probe, and returned to the reservoir, the reservoir is continuously, the exchange chamber volume V_{X} is known and the flow medium exposure time t_{E} can be varied by controlling Q, V_{X}, V_{T} and times t at which the reservoir is sampled.

The instant invention can also be practiced with options including, but not limited to: variable flow rates, including stopping and resting like PMD; changing the flow medium direction (back and forth); omitting the reservoir vessel so all of the flow medium is in the MCRR tubing and exchange chamber(s); an outside-in variation in which the drug or formulation comprises the external medium and the flow medium can be water or simple solution, a dispersion or system including complexing agents or carriers.

To describe the variations, a shorthand notation is used herein as follows. Reservoirs are denoted by R (or Rⱼ if more than one reservoir is present in the setup) and flow pumps are denoted by F (or Fₘ if more than one is used). The exchange chambers are denoted by X, its volume and area by V_{X} and A_{X}, the flow rate through the exchange chamber by Q, and the corresponding external medium by E. (If more than one is present, the corresponding notations are Xₘ, Eₘ, V_{Xm}, A_{Xm} and Qₘ, respectively).

For example, the MCRR flow pattern shown in FIG. 1 is described by the shorthand R-XE-F-R, which denotes that the flow medium is pulled from the reservoir (R) and pulled through the exchange chamber (X) that is immersed in an external reservoir (E), and pushed back by a peristaltic pump (F) to the reservoir (R).

Another embodiment comprises one reservoir, one exchange chamber immersed in an external medium and one pump and is described by the notation R-F-XE-R, which denotes that the flow medium is pulled from the reservoir but pushed through the exchange chamber that is immersed in an external medium and back to the reservoir.

Another embodiment comprises one reservoir and two exchange chambers, each immersed in its own external medium, and connected in series. This is described by the notation R-X₁E₁-F-X₂E₂-R, denoting flow in which the flow medium is pulled from a reservoir vessel (R) and through a first exchange chamber immersed in a first external medium (X₁E₁), through a peristaltic pump (F), then pushed through a second exchange chamber immersed in a second external medium (X₂E₂), then back to the reservoir (R).

Another variation employing one reservoir and two exchange chambers in series, each exchange chamber being immersed in a separate external medium, is described by the notation R-X₁E₁-X₂E₂-F-R, denoting a configuration in which the flow medium is pulled through both exchange chambers then pushed back to the reservoir.

Reversing the flow direction would change the shorthand for the setup. For instance, reversing the flow direction would change an R-XE-F-R configuration to R-F-XE-R. Similarly, reversing the flow direction would change an R-X₁E₁-F-X₂E₂-R to R-X₂E₂-F-X₁E₁-R.

Another variation employing one reservoir and two exchange chambers in parallel, each exchange chamber being immersed in a separate external medium and pulled by a separate pump, is shown in FIG. 2 and is described by the notation

$$R - [ \begin{matrix}{X}_{1} {E}_{1} - {F}_{1} - \\ {X}_{2} {E}_{2} - {F}_{2} -\end{matrix} ] - R ,$$

denoting a configuration in which the flow medium is pulled in parallel through a first exchange chamber immersed in a first external medium by a first pump, and also through second exchange chamber immersed in a second external medium pulled by a second pump, with return from both pumps to the single reservoir.

Additional MCRR setups and flow patterns can be employed, such as adding reservoirs, immersing more than one exchange chamber into the same external medium, etc. Such flow patterns would include, for instance, R-X1E1-F-X2E1-R, denoting flow with two exchange chambers both immersed in the same external medium, and

$$R - [ \begin{matrix}{X}_{1} {E}_{1} - {F}_{1} - \\ {X}_{2} {E}_{2} - {F}_{2} -\end{matrix} ] - R ,$$

denoting flow through two exchange chambers in parallel, each with a separate pump but both immersed in the same external medium. Using additional pumps would allow for different flow rates or flow patterns through the exchange chambers, and it is also possible to vary the flow rates and directions. In addition, different external media would allow different exchange conditions (pH, osmolality, volumes, excipient concentrations) or provide additional components to exchange (salts, reactants, etc.)

The MCRR can also be set up using two reservoirs. For example, FIG. 3 shows a setup described by R1-XE-F-R2, in which the flow medium is pulled from the first reservoir (R1) through the exchange chamber that is immersed in an external medium (XE) then pushed into a second reservoir (R2). Reversing the flow direction would result in the configuration R2-F-XE-R1.

Another two-reservoir configuration is described as R1-X1E1-F-X2E1-R2, in which the flow medium is pulled from the first reservoir (R1) through the first exchange chamber that is immersed in a first external medium (X1E1) then pushed through the second exchange chamber that is immersed in a second external medium (X2E2) into a second reservoir (R2). Reversing the flow direction creates an R2-X2E2-F-X1E1-R1 configuration, in which the flow medium is pulled from the second reservoir (R2), through the second exchange chamber that is immersed in a second external medium (X2E2), then pushed through the first exchange chamber that is immersed in a first external medium (X1E1) and into the first reservoir (R1).

The number of exchange chambers in the probe would be part of the test design, as would be the duration of the test and sampling times

An embodiment in which there is as reversing or back-and-forth flow between two reservoirs (the flow medium is moved from the first to the second reservoir, then moved from the second to the first reservoir, etc.) is described using double-arrows in the notation as [R1-X1E1-F-X2E1-R2]↔[R2-X2E2-F-X1E1-R1]. In a preferred embodiment, all of the flow medium is initially in the first reservoir R1, then it would all be caused to flow to the second reservoir R2, then it subsequently be caused to flow back to R1. The rate of flow from R1 to R2 and R2 to R1 can be the same or different, and the back-and-forth pattern can be repeated any number of times. Sampling can be done at any time from R1 and R2, or even both at the same time.

Typically, the total volume of the flow medium (in the MCRR probe and reservoir) would reduce with each sample taken, which would be used with the flow medium sample concentration data to calculate the mass of the drug gained or lost as a function of the time spent in the exchange chamber by each small volume element of the flow medium. However, it is withing the scope of the instant invention to replace sample volume withdrawn from the reservoir with any liquid medium of known composition, this keeping the total volume constant throughout the test.

The number of exchange chambers and reservoirs is limited only by practical considerations. In addition, the flow rate Q can be constant, varied with time, be intermittent, or change directions. In a preferred embodiment, however, there is one reservoir with one or two exchange chambers, and the flow rate is constant, continuous and in one direction.

The time spent in the exchange chamber for a volume element of the flow medium in a single pass to for a single pass can be calculated or experimentally determined, so the total time spent in the exchange chamber by the flow medium can be determined from the exchange chamber geometry, the flow rate and the time spent flowing, and the extent of the drug that is released or accumulated by the flow medium can be determined as a function of the time spent in the exchange chamber by the flow medium. Since the drug exchange with the external medium for a portion of the flow medium only occurs while that portion is in the exchange chamber, the rate of drug exchange between the flow medium and external medium at a particular time can be determined by taking the slope of the extent of drug gain or loss by the flow medium vs. the time spent in the exchange chamber at said time. (For instance, taking the slope of the plot in FIG. 4 or FIG. 7.)

### DESCRIPTION OF THE FIGURES

FIG. 1 shows a schematic diagram of an embodiment comprising one exchange chamber in an R-XE-F-R configuration.

FIG. 2 shows a schematic diagram of an embodiment comprising one reservoir and two exchange chambers in parallel in an

$$R - [ \begin{matrix}{X}_{1} {E}_{1} - {F}_{1} - \\ {X}_{2} {E}_{2} - {F}_{2} -\end{matrix} ] - R ,$$

configuration.

FIG. 3 shows a schematic diagram of an embodiment comprising one exchange chamber between two reservoirs in an R₁-XE-F-R₂ configuration.

FIG. 4 shows the percent released vs. time for release from an ibuprofen solution in Example 4.

FIG. 5 shows the percent released vs. exposure time for release from an ibuprofen solution in Example 4.

FIG. 6 shows a plot of ln (C/C₀) vs. the exposure time for release from an ibuprofen solution in Example 4.

FIG. 7 shows the percent released vs. time for release from a cyclosporine emulsion in Example 5.

FIG. 8 shows the percent released vs. exposure time for release from a cyclosporine emulsion in Example 5.

FIG. 9 shows shows a plot of ln (1-fraction released) vs. the exposure time for release from a cyclosporine emulsion in Example 5.

### DEFINITIONS OF TERMS

As used herein, the following terms are described. In most cases, the intended meaning is consistent with the usual meaning as understood by one of skill in the art. However, they are defined or described below for completeness or definiteness within the context of the disclosure of the instant invention.

The term “MCRR probe” refers to an assembly of one or more exchange chambers connected by impermeable tubing.

While the term “drug” is used to describe most of the applications and some preferred embodiments, the instant invention is not limited to drugs and includes other chemicals or agents. The term “agent” refers to any chemical that can dissolve and exchange between the flow medium and donor by passage across a membrane, which can include drugs, excipients, and other components of the flow medium or external medium. As used herein, the term “drug” will refer to drugs and other agents unless otherwise noted.

A “formulation” comprises a material or mixture of materials that has optionally been processed by the technique of the instant invention and/or techniques known in the art, and which may optionally include excipients, to produce a final form of a material or mixture with intended properties or characteristics.

The term “flow medium” refers to the liquid medium that is moving through the interior of the MCRR probe, and may be a solution, suspension, emulsion, or otherwise suitably flowable material. The flow medium is contained in the exchange chamber(s), reservoir(s) and connecting tubing.

The term “external medium” refers to any medium outside the probe and in which the exchange chamber of the probe is sufficiently immersed to allow the exchange of drug molecules between the flow medium and the external medium. The external medium is preferably a liquid (solution, suspension, emulsion, micellar medium) or semisolid. The external medium may also be stirred or unstirred, heated or cooled.

The term “exchange chamber” refers to the interior of the portion of the MCRR probe that comprises the permeable membrane that is immersed to be in contact with the external medium and contains the portion of flow medium that is exchanging material with the external medium at any chosen time. The “exchange chamber volume” V_{X} is the volume of the exchange chamber, and the “exchange chamber area”) A_{X} is the area of the exchange chamber that is in contact with the external medium. If more than one exchange chamber is present, each will be denoted with a subscript m as V_{X,m} and A_{X,m}, respectively. As used herein, “immersed to be in contact with the external medium” refers to as immersing an exchange chamber in an external medium sufficiently so exchange of a drug or agent can occur between the flow medium and external medium.

The “exchange chamber inlet,” “probe inlet,” “inlet tubing,” “inlet segment,” or “inlet” refer to the impermeable tubing that connects the source to the exchange chamber. The “probe outlet,” “outlet tubing,” “outlet segment,” or “outlet” refer to the impermeable tubing on the side of the exchange chamber furthest form the source and from which the flow medium exits for collection.

The term “permeable” refers to a membrane or other material that allows drug molecules or other agents to pass by diffusion, convection, etc. In a typical embodiment, permeable membranes are made of impermeable material such as cellulose derivatives into which pores have been introduced, such that the pores can fill with liquid to serve as a diffusion medium between the flow medium and external medium. However, permeable may also refer to allowing the passage of gas molecules.

The term “MCRR probe” refers to an assembly of one or more exchange chambers connected by impermeable tubing.

The term “withdraw” refers to pulling flow medium in a backward direction, which refers to the direction away from the exit end and toward the source end.

The “flow rate” or “volume flow rate” Q refers to the volume per time of flow medium movement in any direction through an exchange chamber. If more than one exchange chamber is present, the flow rate through each will be denoted with a subscript m as Qₘ.

The “time” or “clock time” t takes on its customary meaning and refers to the elapsed time or time interval after some starting timepoint, such as the start of a test or time interval after an event such as withdrawing a volume or mass, etc.

The “transit time” t_{Q} is the average time that a volume element of the flow medium spends in a given exchange chamber during a single pass through it and is given as t_{Q}=V_{X}/Q. If there is more than one exchange chamber, the transit time for each will be denoted by an additional subscript m as t_{Q,m}=V_{X,m}/Qₘ.

The “total volume” V_{T} of the flow medium is the volume in the MCRR probe (exchange chamber plus all tubing) plus the volume in the reservoir vessel at any time. During any time interval “j” in a test, the total volume of the flow medium is denoted by V_{T,j} or V_{T}(j). The initial total volume (at the start of a test) is denoted as V_{T,l} or V_{T}(l). l.

The “reservoir chamber” or “reservoir vessel” is the vessel containing the formulation that is not in the exchange chamber or tubing. The formation “reservoir volume” V_{RES} is the volume of formulation in the reservoir vessel at any specified time.

The term “exposed volume” V_{E} refers to total volume of formulation or flow medium that has passed through the exchange chamber in time t and is given as V_{E}=Q×t. Physically, V_{E} can include flow medium volume elements that pass through the exchange chamber more than once or have not yet passed through the exchange chamber. Thus, V_{E} can be less than, equal to or greater than the total volume V_{T}

The terms “exposure time” or “average exposure time” or “flow medium exposure time” or “formulation exposure time,” denoted as t_{E}, refer to the average time that any volume element of the flow medium has spent in the exchange chamber. If there is only one exchange chamber, it is given as [the volume of flow medium that has passed through the exchange chamber at time t] times [the transit time] divided by [the total volume of the flow medium], or

$${t}_{E} = \frac{Qt}{{V}_{T}} ,$$

$${t}_{Q} = \frac{{tV}_{X}}{{V}_{T}} .$$

. If more than one exchange chamber is present, it is given as the sum over all exchange chambers of each chamber's exposure time, or

$${t}_{E} = {\sum }_{m = 1}^{M} \frac{{tV}_{X , m}}{{V}_{T}} .$$

A “solution” is a molecular dispersion in which a drug or other agent (solute) is dissolved in a solvent. The term “solvent” takes its customary definition as a medium in which the solute is molecularly dispersed and can be a liquid, solid, semi-solid, or gas.

The term “dissolved” refers to the state of a solute mixed in a solvent, in which solute molecules are substantially separated from each other and molecularly dispersed in the solvent. The solvent can be a liquid or solid. In turn, the term “undissolved” refers to the physical state of the solute material in which the solute molecules are not molecularly dispersed in the solvent, but instead are aggregated to primarily interact with themselves. The undissolved form of a solute is most typically a solid but may also be a liquid or gas.

As used herein, the term “dissolution” refers to the transfer of molecules from a physical arrangement or state in which they are undissolved to one in which they are dissolved in a solvent.

As used herein, the term “transfer” refers to movement of molecules of a drug or other agent from a medium containing the drug or agent to another medium that may be void of said drug, or contain a different amount, concentration, or form of the drug.

As used herein, the term “exchange” refers to the transfer of a drug or other agent between a flow medium in an exchange chamber and the external medium in which the exchange chamber is immersed.

As used herein, the term “release” refers to the loss of a drug or other agent from a flow medium to an external medium by diffusion through the permeable membrane while the flow medium is in the exchange chamber. A “release test” measures the loss of drug from a flow medium donor into an external medium, unless otherwise specified. A “release profile” refers to the quantity of drug or agent lost (by diffusion out of the flow medium) vs. time t, where the quantity released can be expressed as the mass (or mass per exchange chamber area), the percent of mass lost (or percent per exchange chamber area), the change in concentration (or change in concentration per exchange chamber area), etc. The release profile may also be expressed as quantity released vs. t_{E} (the formulation exposure time).

As used herein, the term “accumulation” refers to the amount of a drug or other agent gained by the flow medium or exchange medium and is context dependent.

The term “uptake” refers to the gain or accumulation of a drug or other agent by a flow medium from an external medium that acts as a donor by diffusion through the permeable membrane while the flow medium is in the exchange chamber. An “uptake test” measures the gain of drug by a flow medium from an external medium, unless otherwise specified. An “uptake profile” refers to the quantity of drug or agent gained by the flow medium vs. time t or flow medium exposure time t_{E}, where the quantity gained can be expressed using units described with regard to release profiles.

The “bound” or “complexed” refers to the state in which a drug or agent that is bound to or complexed with an agent such as a polymer or protein. The term “carrier” or “complexing agent” refers to an agent that to which a drug or agent molecule can bind or complex. The term “free” or “free and dissolved” refers to the state in which a drug or agent molecule is dissolved in a medium and not bound or complexed to a polymer or protein.

As used herein, the term “redistribution” refers to the transfer of drug molecules into a solution (dissolved form) by dissolution, diffusion, or other process, out of globules or micelles, or away from a bound or complexed state with carriers or complexing agents, such as polymers or proteins, to the free, unbound state. It can also refer to transfer in the reverse direction, from dissolved in a medium into oil globules, micelles, precipitated to undissolved particles, or forming complexes with polymers, proteins, etc.

The term “freeing” or “unbinding” refers to the redistribution of a drug or agent from the bound or complexed state to a state in which the molecule is dissolved and free in a solvent medium.

The term “dispersed phase” refers to discrete structures that are dispersed in a continuous phase. Such structures include, but are not limited to, undissolved particles that are suspended in the continuous medium, micelles or oil globules in an emulsion, carriers, and binding agents such as polymers that may be dissolved, liposomes, cyclodextrins, etc. Of particular interest are dispersed phases that contain some drug, such as drug particles in emulsion or suspension, or drug molecules that are partitioned into micelles or emulsion globules or bound to proteins and polymers. (The continuous phase is most often aqueous, but this is not required by the invention.)

The term “donor” refers to the medium that is losing the drug to the other medium, and the term “receiver” refers to the medium that is accepting the drug.

The term “diffusible” means able to diffuse in or through a medium and preferably a liquid or semisolid medium.

The term “contained” means the drug is present in some form in the medium, such form including but not limited to the following: drug that is dissolved in the medium; drug that is suspended as undissolved or partially dissolved particles in the medium; drug that is bound to or complexed with proteins, polymers, or other complexing materials; drug that is dissolved, partitioned or otherwise distributed in distinct phases such as oils, surfactants, emulsions, micelles, liposomes, etc.

The term “perturbation” means a change or altering of one or more properties of the flow medium or external medium that induce at least one physical process in response. The term “perturb” means to bring about a perturbation.

### DISCUSSION OF THE INVENTION

The approach taken by the instant invention to solving the problems associated with the previous methods is the use of a novel back-and-forth or pull-push flow pattern using a low volume flow medium. The exchange chamber has a particularly high area-to-volume ratio, which allows detecting changes in concentration even when small amounts of drug mass are gained or lost by the flow medium. This is because the rate at which drug molecules cross the exchange chamber membrane is proportional to the surface area A_{X}, while the change in drug concentration is proportional to the volume of flow medium in the exchange chamber V_{W}. Thus, increasing the ratio A_{X}/V_{X} results in greater sensitivity of the concentration to the drug that exchanged across the exchange chamber membrane with the external medium.

The drug exchange between the flow medium and external medium occurs by diffusion and involves only those drug molecules that are dissolved and free. In a solution, this would involve all of the dissolved molecules, while in dispersed systems such as emulsions, this involves the molecules that are dissolved and free in the external continuous phase. (To be in the free form, it is assumed that the drug or other agent is not precipitated or undissolved, complexed or otherwise bound to other molecules or particles, not incorporated into micelles, microemulsions, void spaces in particles, etc.)

When drug molecules are added to a solvent and there is no binding, complexation, trapping, precipitation, etc., the total drug concentration should be the same as the free drug concentration. However, for many systems this is not the case. Examples include multiphase systems, such as micelles, nanoemulsions and microemulsions, suspensions containing undissolved drug particles, and drug complexes with proteins, polymers such as cyclodextrin, etc. Most typically, the free drug of interest in the pharmaceutical sciences is in the aqueous phase, into which dissolution or other redistribution occurs.

The following discussion focuses on systems in which the free drug is in the aqueous phase of a formulation or medium, which is typically of pharmaceutical interest. However, the same or similar principles could apply to other types of diffusing medium in which the free drug is considered—for instance, oil is the continuous phase in a w/o (water-in-oil) emulsion. The quantitative gain or loss of drug molecules by the flow medium due to exchange with an external medium is determined by the exchange chamber area A_{X} and the diffusion rate across the membrane comprising the exchange chamber. In turn, the drug diffusion across the MCRR exchange chamber membrane depends on the free drug concentration difference across the membrane. For systems in which not all of the drug is free, this depends on the fraction of the drug that is free in the donor medium.

When the donor is the external medium (the external medium comprises a formulation being tested), the donor volume is typically much larger than the MCRR exchange chamber volume V_{X} (tens to hundreds of mL vs. 1 to 10 μL). As a result, uptake of the drug by flow medium received from an external medium is typically small enough to avoid a meaningful change in mass balance in the donor, and does not induce a redistribution of the drug in the donor to the free form in the aqueous phase (e.g., by dissolution, release of complexed drug, release of drug partitioned into micelles or emulsions, etc.) Thus, MCRR using an external medium as the donor is particularly well suited for testing properties of a medium that do not change due to drug redistribution. This is advantageous for testing equilibrium configurations, or testing media for which the properties change due to factors other than drug redistribution, including chemical reactions, kinetic displacement of drugs from complexing agents due to dilution, slow drug partitioning, drug precipitation out of supersaturated solutions, etc.

In contrast, when the drug is in the flow medium, the donor volume can be much smaller than the receiver volume. In this setup, it is possible that drug redistribution into the free form can occur due to the loss of free drug from the flow medium to the external medium. This can occur by dissolution, release from micelles or emulsions, release from binding or complexing agents, etc., and the rate of change of the amount of drug in the flow medium can be strongly dependent on the balance of the rate of redistribution vs. the rate of loss to the receiver by diffusion across the exchange chamber membrane. Thus, MCRR that is done using the flow medium as the donor can be used as a test to characterize the drug loss or release, which in turn can provide information about the redistribution processes. This applies for any microdialysis method, including conventional continuous flow microdialysis, pulsatile microdialysis (PMD), and MCRR.

Examples of pharmaceutical interest include, but are not limited to, release or dissolution of drugs from topical ophthalmic products such as emulsions and suspensions. However, many of these products contain excipients such as polymers and viscosity-enhancing agents, which can reduce the sensitivity of CFMD and PMD (i.e., show less increase in drug loss per increased time spent by the flow medium in the exchange chamber), especially with slow flow rates (for CFMD) or longer resting times (for PMD), presumably due to alterations in the liquid medium structure, such as gelling, etc.

However, the non-constant flow patterns of MCRR, including reversing the flow, surprisingly can reduce these effects and increase the sensitivity. This is believed to be because MCRR can increase the time spent in the exchange chamber by employing more cycles instead of using slow flow rates or long resting times. In addition, the flow patterns disrupt any gelation or other structure-type formation in the liquid flow medium. Reversing flow patterns may also create some agitation in the exchange chamber that would not occur with a constant flow pattern, or a simple push-stop-push pattern. Without intending to be bound by any model or theory, such agitation may have the benefit of increasing dissolution rates from suspended particles, in a manner analogous to stirring vs. not stirring during particle dissolution testing (for instance, in a USP 1-2). For drugs with poor aqueous solubility, such as are typical for ophthalmic suspensions, this can prove useful when assessing drug dissolution.

Further, when the flow medium is the donor, the drug release occurs by loss of the free drug from the flow medium by diffusion or other transport across a membrane into the external medium, which occurs with simultaneous dissolution or release of the drug in the formulation (for instance) into the free form, from which more loss by transport across the membrane can occur, etc. Also, because of the membrane, no sudden large flow medium dilution factor is introduced.

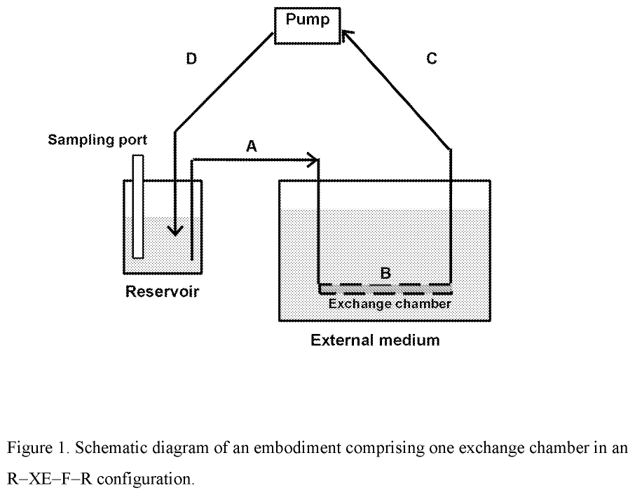

In its simplest generic form, depicted in FIG. 1 (with different but not confusing nomenclature), is an apparatus of the instant invention comprising a first holding means for holding a liquid containing as a component at least one poorly-soluble material, said first holding means communicating with at least one second holding means for holding a liquid received from said first holding means and in said second holding means there being immersed a separation means for separating at least one of the components of said liquid, said second holding means communicating with said first holding means in a manner by which to return said liquid to said first holding means in a controllable recycle manner, and a powered transfer means to controllably move said liquid forward through said separation means and backward for said recycle, and any of said holding means optionally containing a sampling means for withdrawing samples from said liquid in said holding means.

Without intending to be bound by any model or analysis, examples are given that illustrate the instant invention and some applications. However, these examples should not be interpreted as limiting the scope or applications of the instant invention.

### Example 1. Description of a Preferred Embodiment for Release of a Drug or Agent from the Flow Medium to the External Medium

An example model is given herein to illustrate one embodiment of MCRR that is useful, comprising one exchange chamber and a single stirred reservoir (R-XE-F-R).

In this example, the flow medium is a formulation composition comprising a drug with an initial concentration C₀ and the external medium is initially void of the drug, and the drug is released from the flow medium (donor) to the external medium (receiver).

The release test setup is shown in FIG. 1. The MCRR probe comprises impermeable polyimide tubing (A) with an opening submerged in the formulation in the reservoir vessel, which is connected to the exchange chamber that is immersed in the receiver medium (B), which is connected to polyimide tubing (C), which is connected to the peristaltic pump, which is connected to polyimide tubing (D) that returns to the reservoir vessel. The flow medium moves out of the reservoir, through the probe from A to D, and back into the reservoir.

In a typical embodiment, illustrated in FIG. 1, the exchange chamber comprises a commercially available tubular segment of permeable regenerated cellulose membrane with a MWCO (molecular weight cutoff) of 13 kD (kilodaltons) and a nominal inner radius a of about 0.01-0.05 cm (for instance, Spectra/Por Hollow Fiber membranes, Repligen, Waltham MA). The length of the exchange chamber is ˜1-15 cm and the volume V_{X} ranges from less than 1 μL to more than 10 μL. (These are typical values and serve as examples but are not meant to limit any ranges that fall within the scope of the invention.)

The reservoir is in a 1-5 mL glass vessel that is sealed and has a sampling port through which a needle can be passed, and the volume of the external medium is typically 250-1000 mL. Samples of volume V_{S} are taken from the flow medium in the reservoir through the sample port using an accurate needle/syringe set. The reservoir is continuously stirred, and the flow through the MCRR probe is maintained at a constant flow rate Q in one direction.

The external medium is also continuously stirred, and its volume is typically ranges from about 10-1000 mL, so it is much greater than V_{X} and preferably much greater than the total volume of the flow medium V_{T}, so sink conditions are maintained in the external medium.

The MCRR test is performed as follows:
a) providing a probe apparatus comprising an exchange chamber (a section of relatively highly permeable membrane relative to any materials to which the membrane is attached) and positioned between an inlet with one end in a reservoir vessel and an outlet back to said reservoir, and through which membrane the diffusible agent is to be transferred), in which said inlet and outlet are substantially impermeable to said diffusible agent;
b) providing a flow medium of initially known total volume comprising a drug composition in which the drug is at least partially dissolved, and an external medium that is substantially void of any drug initially.
c) putting said exchange chamber in contact with said external medium by immersing it sufficiently in the external medium so exchange of said diffusible agent can occur between the flow medium and external medium;
d) initially filling said probe apparatus and at least partially filling said reservoir with said flow medium;
e) causing said flow medium to flow from said reservoir through said exchange chamber and impermeable tubing, and back to said reservoir using a known flow rate Q in one direction, and for a specified period of time;
f) withdrawing a sample of a specified volume from said flow medium at a specified time;
g) determining the concentration and mass of said diffusible agent in said sample of flow medium;
h) optionally, repeating steps (e) through (g) with the same flow rate, direction, and sample volume;
i) calculating the concentration in the flow medium for each sample, and plotting the change of said drug concentration in the flow medium concentration vs. the time t.

The above procedure causes a release of the drug or agent from the flow medium while in the exchange chamber into the external medium by diffusion of dissolved molecules through the pores of the exchange chamber membrane. The release exchange occurs only from those portions of the formulation that are in the exchange chamber at any instant in time (no drug or agent is lost from the flow medium through the impermeable inlet/outlet tubing), so the drug release depends on the time spent in the exchange chamber by the flow medium, which is tracked by the exposure time t_{E}. However, simultaneous redistribution of the drug (e.g., out of the globules and micelles into the aqueous phase, dissolution of undissolved particles, transfer from a complex to free in solution form, depending on the composition and properties of the flow medium) occurs everywhere in the formulation (in the exchange chamber, impermeable tubing, and reservoir) and is a function of the time t.

To perform a MCRR release test, an accurately determined quantity of flow medium (e.g., the drug formulation) is initially loaded into a reservoir vessel and the MCRR probe (exchange chamber and all tubing A-D in FIG. 1). The total volume of the flow medium V_{T} is the sum of the volume in the probe (exchange chamber and tubing) and the volume in the reservoir V_{R}. Typically, the initial V_{T} is about 0.5-3 mL. Samples are taken at specified times, so V_{T} (and the volume in the reservoir V_{R}, since the probe remains full) decrease by V_{S} with each sample. The initial V_{T} and V_{R} and are chosen so the specified number of samples in the test design to be taken without disrupting the reservoir stirring or the MCRR flow from the reservoir, through the probe and back. (Although not discussed in this example, it is possible to replace the volume of each withdrawn by adding an equal volume to the reservoir of a replacement medium of known composition, thus keeping V_{T} and V_{R} constant. This is discussed in Example 3.)

During the MCRR release test, the formulation is continuously withdrawn from the reservoir vessel, circulated through the MCRR probe, and returned to the reservoir vessel at a constant flow rate Q. (A constant flow rate Q is a preferred embodiment, but Q does not have to be constant.) The exchange chamber is immersed in a temperature-controlled, stirred external medium. Also, because the flow medium is either flowing through the MCRR probe or is continuously stirred in the reservoir vessel, it is never at rest, which substantially reduces or eliminates problems seen with viscous liquids or dispersed systems (for instance, viscosity transients, settling and sedimentation, etc. when tested using CFMD or PMD).

When MCRR is performed in this manner, each volume element of the flow medium moves through the exchange chamber at the same flow rate Q and spends the same amount of time in the exchange chamber per pass through it, which is denoted as the transit time and given as t_{Q}=V_{X}/Q. The flow is started at the beginning of the test, corresponding to time t=0. At any time/after the start of the test, the average time each small volume element spends in the exchange chamber is referred to as the average exposure time t_{E}. The differentiation between t_{E} and t (the exposure time and the test elapsed time) is made because t_{E} tracks the time the flow medium spends in the exchange chamber releasing the drug to the receiver medium. On the other hand, if any other processes are going on, such as redistribution or dissolution, they would occur for the entire test time duration, not just when any fraction of the flow medium is in the exchange chamber

It is possible to relate the time and exposure time as follows. During any time interval Δt, the change in the exposure time Δt_{E} is

$$( 1 )$$

$$Δ {t}_{E} = \frac{[ \begin{matrix}volume that has flowed through \\ the exchange chamber during Δ t\end{matrix} ] \times {t}_{Q}}{{V}_{T}} = \frac{[ Q Δ t ] \times [ \frac{{V}_{X}}{Q} ]}{{V}_{T}} = \frac{{V}_{X}}{{V}_{T}} Δ t$$

The act of sampling reduces V_{T} and the quantitative relationship between t_{E} and t must reflect this effect. The following notation will be used. For a test in which J samples are taken, each sample number is denoted by the subscript j=1, 2, . . . . J, and the time at which each sample is taken (the sample times) is denoted as t₁, t₂, . . . tⱼ, . . . t_{J}. The sampling interval Δtⱼ for a given sample j is the time interval leading up to the sample from the previous one and is given as Δtⱼ=tⱼ−tⱼ₋₁. (There are J sampling intervals, and the sampling interval-1 is given as Δt₁=t₁ since the test starts at t=0.) For other properties, subscripts will similarly indicate the sampling interval with which the property is associated. For instance, V_{T,1} denotes the total flow medium volume during sampling interval-1 (from t=0 to t=t₁), V_{T,2} denotes the total volume during sampling interval-2 (from t₁ to t₂), etc. For properties associated with specific times, the times are specified in parentheses. For instance, the concentration at time t₁ is denoted as C(t=t₁).

Eq. (1) prescribes a simple relationship between the exposure time t_{E} of a sample and the time t at which a sample is taken. For each time interval, the elapsed time interval Δtⱼ required to produce a corresponding increase in the exposure time Δt_{E,j} is related to the total volume during the time interval by

$$Δ {t}_{E , j} = Δ {t}_{j} \times \frac{{V}_{X}}{{V}_{T , j}} \qquad ( 2 )$$

$$or$$

$$Δ {t}_{j} = Δ {t}_{E , j} \times \frac{{V}_{T , j}}{{V}_{X}}$$

The sampling intervals are taken as follows. Interval-1 is from the start of the test to the first sample, interval-2 is from immediately after taking the first sample to the second sample, interval-3 is from immediately after taking the second sample to the third sample, and so on. The total flow medium volume during interval-1, V_{T}(l), is the initial total volume, and V_{T} for each subsequent interval is reduced as

$${V}_{T , j} = {V}_{T , 1} - ( j - 1 ) {V}_{S} \qquad ( 3 )$$

The sampling time t_{J}, at which sample J (the last sample) is taken and which corresponds to t_{E,J} is the sum over the J time intervals, given as

$${t}_{J} = {\sum }_{j = 1}^{J} Δ {t}_{j} = {\sum }_{j = 1}^{J} Δ {t}_{E , j} \times \frac{{V}_{T , j}}{{V}_{X}} \qquad ( 4 )$$

$${t}_{E} = {\sum }_{j = 1}^{J} Δ {t}_{E , j} = {\sum }_{j = 1}^{J} Δ {t}_{j} \times \frac{{V}_{X}}{{V}_{T , j}} \qquad ( 5 )$$

$$for$$

$$t = {\sum }_{j = 1} Δ {t}_{j}$$

Table 1 gives an example calculation, with a starting total volume of V_{T,1}=1000 μL, V_{S}=90 μL, Q=40 μL/min (0.6667 μL/sec), and V_{X}=7 μL. In this example, the test design specifies the exposure times and calculating the corresponding time intervals from Eq. (2), then calculating the total time from the start of the test using Eq. (4).

TABLE 1
Example of determining test sampling times.

| Interval | t_{E} | Δt_{E, j} | V_{T, j} | | Δtⱼ | Time tⱼ |
|---|---|---|---|---|---|---|
| j | (sec) | (sec) | (μL) | V_{T, j}/V_{X} | (sec) | (sec) |
| 1 | 20 | 20 | 1000 | 142.9 | 2857 | 2857 |
| 2 | 40 | 20 | 910 | 130.0 | 2600 | 5457 |
| 3 | 60 | 20 | 820 | 117.1 | 2343 | 7800 |
| 4 | 120 | 60 | 730 | 104.3 | 6257 | 14057 |
| 5 | 180 | 60 | 640 | 91.4 | 5486 | 19543 |
| 6 | 300 | 120 | 550 | 78.6 | 9429 | 28971 |
| 7 | 600 | 300 | 460 | 65.7 | 19714 | 48686 |
| 8 | 900 | 300 | 370 | 52.9 | 15857 | 64543 |

The drug release described above occurs solely through the exchange chamber membrane, not from the entire probe. However, as a result of the constant stirring in the reservoir and continuous flow through the probe, the entire volume of each MCRR sample j is assumed to comprise a uniform exposure time t_{E,j}.

In a preferred embodiment, the exchange chamber is a “long” cylindrical. (As used herein, a “long” cylinder is one for which its length L is significantly greater than its inner radius a, so the area-to-volume ratio is A_{X}/V_{X}≈1/a.) The volume of the exchange chamber can vary but the area-to-volume ratio of the exchange chamber is preferably greater than about 10 cm⁻¹, more preferably greater than about 50 cm⁻¹, and even more preferably greater than about 100 cm⁻¹. For a long cylinder, these ratios correspond to a˜2 cm (2000 microns), 0.2 cm (2000 microns) and 0.02 cm (200 microns), respectively.

For a solution at rest in an exchange chamber of inner radius a, it is possible to estimate the time required for the solution to release 99% of the drug contained in a solution to the external medium, denoted by t*. This time is of interest because it relates the properties of the exchange chamber and the timeframe of the drug release from a solution, which can serve as an approximate estimate of the timeframe for a drug to release from the aqueous phase of the flow medium while in an exchange chamber.

The parameter t* is of interest because it is an indicator of the release timeframe. For shorter t*, less time is required to release a given fraction of drug that is dissolved in a solution as a flow medium, and analogous conclusions apply to the aqueous phase of other compositions such as emulsions, suspensions, etc. Thus, an exchange chamber with a shorter t* will be more able to distinguish fast vs. slow processes, such as release from the aqueous phase vs. slower redistribution processes.

The equations below calculate t* for a solution at rest in the exchange chamber, but still are a useful guide to evaluating the timeframe for release for a flow medium in MCRR tests. Following the calculations of Kabir et al. (2005),

$${t}^{*} \geq \frac{4.61 {a}^{2}}{{β}^{2} D} ,$$

where D is the diffusion coefficient of the particular drug in the solution, β is the first (lowest positive) root of the equation βJ₁(β)=λJ₀(β), where J₀ and J₁ are Bessel functions of the first kind of order 0 and 1, respectively, λ=aP/D, and P is the exchange chamber membrane permeability with respect to the particular drug. While not necessary to practice the invention, it is preferred to employ exchange chambers comprising porous membranes of impermeable materials into which pores have been introduced, such as tubular membranes of cellulose derivatives or other materials. For such membranes, the drug and other agents exchange with the external medium by diffusing through the liquid-filled membrane pores, and the approximation can be made that

$$P = \frac{a ε}{τ h} ,$$

where ε is the membrane porosity (the fraction of the total membrane volume that is “empty” or pores), and τ is the average pore tortuosity (the ratio of pore pathlengths to the thickness of the membrane h). One of skill in the art would recognize that the permeability of the exchange chamber membrane can be increased by increasing the porosity ε, or decreasing the tortuosity τ or membrane thickness h.

For a given exchange chamber and drug solution, t* will increase as the exchange chamber inner radius increases or β decreases. Mathematically, for a given a, β decreases if λ decreases, which occurs as a result of decreasing P. Some calculated values of t* are listed in Table 2, using high and low exchange chamber parameters for the membrane permeability and radius and a diffusion coefficient value of D=5×10-6 cm/s, which is a representative value for any drugs in water. [Kabir et al, 2005]

TABLE 2
Values of t* for various exchange chamber membrane
permeabilities P and radii a, using a diffusion
coefficient value of D = 5 × 10⁻⁶ cm²/s

| P (cm/s) | a cm | λ (no units) | β (cm) | t* (sec) |
|---|---|---|---|---|
| 3.00E−04 | 0.01 | 0.600 | 1.013 | 69 |
| 3.00E−04 | 0.10 | 6.000 | 1.828 | 2109 |
| 1.00E−05 | 0.01 | 0.020 | 0.340 | 610 |
| 1.00E−05 | 0.10 | 0.200 | 0.720 | 13597 |

When selecting materials for the exchange chamber membrane, two factors are of most interest—the inner exchange chamber radius and the membrane permeability. As a practical matter, the inner radius is often chosen based on what is available in the market, but it is possible to manufacture to custom radii if desired. The permeability is a function of the membrane thickness as well as its porosity and tortuosity. While it is also possible (if not practical) to manufacture to specifications, the typical procedure is to select exchange chambers based on the molecular weight cutoff (MWCO, expressed in daltons) of the pores. Larger MWCOs typically mean larger diameters and lower tortuosity values. In addition, larger pores tend to produce higher permeabilities, presumably because the porosity is higher (larger fraction of the membrane is pore space) or because there is more room for diffusing molecules to migrate and less colliding with the pore walls.

Preferably, the exchange chambers comprise inner radii of about ≤0.1 cm (10,000 microns) and a MWCO not exceeding about 300 kD (300,000 daltons), and more preferably a≤about 0.02 cm and a MWCO not exceeding about 100 kD. In a particularly practical embodiment, commercially available tubular membranes are used for which a is less than about 0.015 cm and the MWCO is less than about 20 kD.

In practice, once a is known, the exchange chamber volume V_{X} is determined by its length L, which typically varies from about 1 to about 20 cm. Generally, for a given flow rate Q and exchange chamber radius, a larger V_{X} means more exchange of the drug between the flow medium and external medium per pass through the exchange chamber.

When sampling the reservoir in the MCRR test, the volume of each sample taken from the reservoir V_{S} must be sufficient to allow an accurate HPLC analysis. Also, since the total flow medium volume reduces by V_{S} every time a sample is taken, the initial total flow medium volume must large enough so the volume in the reservoir is always sufficient to allow stirring and facilitate uninterrupted flow of the flow medium (unless the pump is stopped as part of the test design). The instant invention can also be practiced by replacing the sample volume, thus keeping the total flow medium volume constant. However, this is not a preferred practice since it introduces another source of change into the flow medium being tested.

### Example 2. Description of a Preferred Embodiment for Uptake of a Drug or Agent by the Flow Medium from the External Medium

An example is provided herein for another use of the setup detailed in Example 1, where the MCRR setup is employed in an uptake test in which the flow medium accumulates drug from the external medium. As with Example 1, the MCRR setup comprises one exchange chamber and a single stirred reservoir (R-XE-F-R configuration), and a stirred external medium that is preferably, but not necessarily, much larger than the volume of the exchange chamber or the total volume of the flow medium.

In this example, the flow medium is initially void of the drug, and the external medium comprises a drug that is at least partially dissolved and the drug is taken up by the flow medium (receiver) from the external medium (donor). The external medium may be a simple solution or a complex composition such as an emulsion, suspension or drug-carrier complex. The flow medium may be a water or buffer or other solution, or an emulsion or solution comprising a protein, polymer or other complexing agent, or an agent that chemically reacts with the drug or agent taken up by the flow medium.

In this example, the reservoir is continously stirred. The external medium is also continuously stirred, and its volume may or may not be large compared to the volume of the exchange chamber.

The MCRR test is performed as follows:
a) providing a probe apparatus comprising an exchange chamber (a section of relatively highly permeable membrane relative to any materials to which the membrane is attached) and positioned between an inlet with one end in a reservoir vessel and an outlet back to said reservoir, and through which membrane the diffusible agent is to be transferred), in which said inlet and outlet are substantially impermeable to said diffusible agent;
b) providing an external medium comprising a drug composition in which the drug is at least partially dissolved, and a flow medium of initially known total volume that is substantially void of any drug initially.
c) putting said exchange chamber in contact with said external medium by immersing it sufficiently in the external medium so exchange of said diffusible agent can occur between the flow medium and external medium;
d) initially filling said probe apparatus and at least partially filling said reservoir with the flow medium;
e) causing said flow medium to flow from said reservoir through the exchange chamber and impermeable tubing, and back to said reservoir using a specified flow rate Q in one direction, and for a specified period of time;
f) withdrawing a sample of a specified volume from said flow medium at a specified time;
g) determining the concentration and mass of said diffusible agent in said samples of flow medium;
h) optionally, repeating steps (e) through (g) with the same flow rate, direction, and sample volume;
i) calculating the concentration in the flow medium for each sample, and plotting the change (gain or loss) in concentration vs. the time t.

The above procedure causes an uptake of the drug by the flow medium while in the exchange chamber from the receiver medium by diffusion of dissolved molecules through the pores of the exchange chamber membrane. The uptake exchange occurs only from those portions of the formulation that are in the exchange chamber at any instant in time (no drug is lost from the formulation through the impermeable inlet/outlet tubing), so the drug uptake depends on the time spent in the exchange chamber by the flow medium, which is tracked by the exposure time t_{E}. However, if the flow medium comprises other phases, complexing agents or agents that chemically react with the drug, there can be a simultaneous redistribution of the newly accumulated drug from the aqueous continuous phase (which acts to receive the drug molecules from the external medium) to globules, micelles, or to a bound or complexed form. This redistribution can occur everywhere in the flow medium (exchange chamber, impermeable tubing, and reservoir) and is a function of the time t.

The flow medium may also comprise an agent that chemically reacts with the drug or agent taken up by the flow medium from the external medium for purposes including, but not limited to, increasing analytical sensitivity to the drug or agent, measuring a chemical reaction affinity or rate, etc.

During the MCRR uptake test, the formulation is continuously withdrawn from the reservoir vessel, circulated through the MCRR probe, and returned to the reservoir vessel at a constant flow rate Q . (As noted in Example 1, constant Q is one a preferred embodiment, but it is not necessary that Q be constant.) The exchange chamber is immersed in a temperature-controlled, stirred external medium. Also, the flow medium is either flowing through the MCRR probe or is continuously stirred in the reservoir vessel, so it is never at rest. In a preferred embodiment, the exposure times are selected as part of the MCRR test design and the sampling times are calculated using Eq. (4).

It would be apparent to one of skill in the art that, if the volume of the external medium is much larger than V_{X} and V_{T}, the loss of the drug from the external medium due to flow medium uptake causes a very small fractional change in the drug content and concentration in the external medium that would not induce substantial redistribution among any phases or complexes that might be present in the external medium. Such a setup would be useful for evaluating the effects of dispersed components or complexing carrier agents if present in the flow medium. One of skill in the art would also recognize that the external medium composition and properties (pH, temperature, etc.) can change during the course of an MCRR uptake test.

### Example 3. Description of a Preferred Embodiment for Release of a Drug or Agent from the Flow Medium to the External Medium with Sample Volume Replacement

An example model is given herein to illustrate one embodiment of MCRR that is useful, comprising one exchange chamber and a single stirred reservoir (R-XE-F-R). The MCRR setup is the same as described in Example 1 with one difference—after sampling, the volume V_{S} of sample removed from the reservoir is replaced by an equal volume of a replacement fluid that contains no drug or, optionally, other agent. The replacement fluid could be water, an emulsion made the same way as an emulsion being tested but containing no drug, etc.)

The sample is withdrawn from the reservoir and the replacement medium is added into the reservoir immediately after withdrawing the sample. The reservoir is continuously stirred, and the flow through the MCRR probe is maintained at a constant flow rate Q in one direction. The external medium is also continuously stirred.

The MCRR test is performed as follows:
a) providing a probe apparatus comprising an exchange chamber (a section of relatively highly permeable membrane relative to any materials to which the membrane is attached for support) and positioned between an inlet with one end in a reservoir vessel and an outlet back to said reservoir, and through which membrane the diffusible agent is to be transferred);
b) providing a flow medium of initially known total volume comprising a drug composition in which the drug is at least partially dissolved, and an external medium that is substantially void of any drug initially.
c) putting said exchange chamber in contact with said external medium by immersing it sufficiently in the external medium so exchange of said diffusible agent can occur between the flow medium and external medium;
d) initially filling said probe apparatus and at least partially filling said reservoir with the said flow medium;
e) causing said flow medium to flow from said reservoir through the exchange chamber and impermeable tubing, and back to said reservoir using a specified flow rate Q in one direction, and for a specified period of time;
f) withdrawing a sample of a specified volume from said flow medium at a specified time, and replacing said sample volume with an equal volume of a replacement fluid that is void of said drug;
g) determining the concentration and mass of said diffusible agent in said samples of flow medium;
h) optionally, repeating steps (e) through (g) with the same flow rate, direction, and sample volume;
i) calculating the concentration in the flow medium for each sample, and plotting the change (gain or loss) in concentration vs. the time t.

The above procedure causes a release of the drug or agent from the flow medium while in the exchange chamber into the external medium by diffusion of dissolved molecules through the pores of the exchange chamber membrane. The release exchange occurs only from those portions of the formulation that are in the exchange chamber at any instant in time (no drug or agent is lost from the flow medium through the impermeable inlet/outlet tubing), so the drug release depends on the time spent in the exchange chamber by the flow medium, which is tracked by the exposure time IE. However, simultaneous redistribution of the drug (e.g., out of the globules and micelles into the aqueous phase, dissolution of undissolved particles, transfer from a complex to free in solution form, depending on the composition and properties of the flow medium) occurs everywhere in the formulation (in the exchange chamber, impermeable tubing, and reservoir) and is a function of the time t. In addition, there is a dilution of the flow medium introduced after each sample is withdrawn due to the addition of the replacement fluid. Such dilution could be with respect to the drug only or with respect to all components of the flow medium, depending on the composition of the replacement fluid.

When MCRR is performed in this manner, the total volume does not reduce with sampling so V_{T} is constant throughout the test and equals what corresponds to V_{T,1} in Example 1. As a result, at any time/after the start of the test, the average time each small volume element spends in the exchange chamber, referred to as the average exposure time t_{E}, is obtained by modifying Eq. (1) and Eq. (4) relating the time to the exposure time as

$${t}_{E} = \frac{{V}_{X}}{{V}_{T}} t , \qquad ( 6 )$$

The time at which sample j is taken is related to the exposure time of sample j becomes

$${t}_{j} = \frac{{V}_{T}}{{V}_{X}} {t}_{E , J} \qquad ( 7 )$$

The drug release described above occurs solely through the exchange chamber membrane, not from the entire probe. However, as a result of the continuous stirring in the reservoir and continuous flow through the probe, the entire volume of each MCRR sample is assumed to comprise a uniform exposure time t_{E,j}.

### Example 4: Release of Ibuprofen from a Flow Medium Solution to an External Medium

In the examples, the release test setup described in Example 1 (R-EX-F-R configuration) was used with the following MCRR probe properties. Each MCRR probe comprised an exchange chamber that was made of regenerated cellulose with a MWCO of about 13 kD, with polyimide tubing segments as the impermeable inlet (about 10 cm) and outlet (about 10 cm). The exchange chambers were characterized using previously disclosed methods [Bellantone, 2012] for the volume V_{X} and membrane permeability relative to ibuprofen. The external medium was 500 mL of 25 mM phosphate buffer at pH 7 and 20° C.

An aqueous solution was prepared, containing ibuprofen 20 μg/mL in a phosphate buffer solution (25 mM and pH=2.5-3), which was used as the flow medium. A second, identical, buffer but void of the ibuprofen was also prepared as external medium.

A release test was performed using the ibuprofen solution as the flow medium (donor), as instructed below:
a) Immerse the MCRR probe exchange chamber sufficiently in a stirred aqueous external medium so exchange of said diffusible agent can occur between the flow medium and external medium;
b) Pump the flow medium through the MCRR probe at a constant Q;
c) Collect samples from the reservoir at times corresponding to selected exposure times, where the times were calculated from the exposure time and the test setup;
d) Repeat steps (b) and (c) until all samples are collected.
e) Perform an appropriate assay (HPLC, etc.) on each collected sample to determine the concentration and mass of ibuprofen in each sample;
f) Plot the percent of ibuprofen released vs. the time and the exposure time.

The release test was performed in triplicate, using three MCRR setups to perform one test each. Each setup comprised one MCRR probe, one reservoir, and one external medium. The nominal setups were the same for each test (within experimental setup error). In each setup, reservoir and external medium were both continuously stirred.

The MCRR setup was as follows:
Q=0.040 mL/min (0.000667 mL/sec)
V_{T,1}=0.604 mL (average of the V_{T,l} for the three MCRR test setups)
V_{S}=0.040 mL/sample
V_{X}=0.00768 mL (average of V_{X} for the three exchange chambers used in the tests)
Flow medium: ibuprofen in 25 mM phosphate buffer at pH 7
Initial flow medium ibuprofen concentration=25.0 μg/mL
External medium=1000 mL of 25 mM phosphate buffer pH=7 at 20° C.
Sample exposure times: 15, 30, 60, 120, 180, 300 seconds.
Sample times: 19.2, 37.1, 70.5, 132.0, 188.5, 291.3 minutes (average of the calculated sample times corresponding to the sample exposure times, taken over the three MCRR test setups)

The release data are derived from the fraction of the initial concentration remaining, given by Eq. (8), and the percent of the concentration lost relative to the initial concentration in sample j, given by Eq. (9), as

$$Fraction of the concentration remaining = \frac{C ( t = {t}_{j} )}{C ( t = 0 )} = \frac{C}{{C}_{0}} \qquad ( 8 )$$

$$Percent of concentration released = 100 [ 1 - \frac{C ( t = {t}_{j} )}{C ( t = 0 )} ] \qquad ( 9 )$$

Since the flow medium is a solution with no dispersed structures or complexing agents, there is only one process (release) and no response (the mixing of the solution volume elements is very fast compared to release and approximated as being instantaneous, which is a standard assumption for well-stirred liquids).

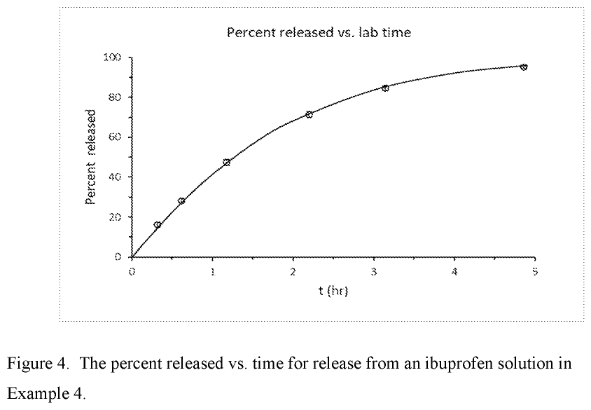

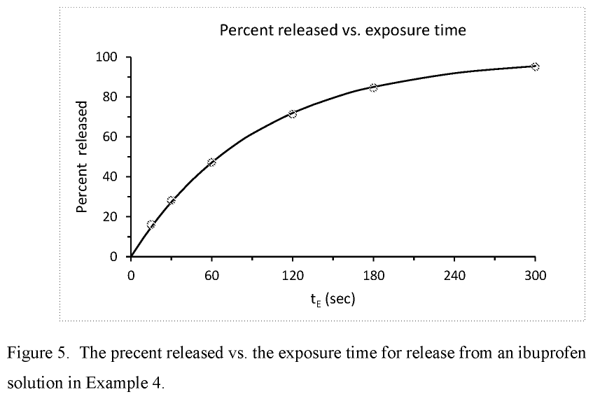

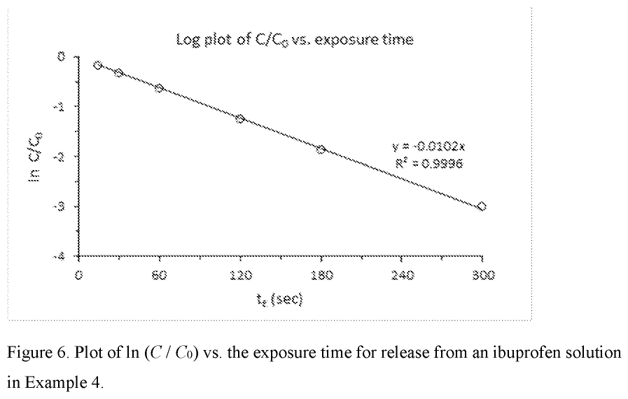

FIG. 4 shows the percent released vs. the time and FIG. 5 shows the percent released vs. the exposure time. The percent released approached 100% and the two plots appear visually similar in shape. However, the test time of nearly five hours (291.3minutes) corresponded to exposure times of up to five minutes (300 seconds). FIG. 6 shows a log plot of In (1-fraction released) vs. the exposure time, which is linear and consistent with the theoretical equation

$$C = {C}_{0} exp ( - \frac{QE}{{V}_{X}} {t}_{E} ) or ln \frac{C}{{C}_{0}} = - \frac{QE}{{V}_{X}} {t}_{E} \qquad ( 10 )$$

where E is the extraction ratio that equals the fraction of the dissolved drug that is released from a volume element of the flow medium on passing through the exchange chamber. For this release test, a value of E=0.117 is calculated from Eq. (10) using the slope of −0.0102 from FIG. 5, the average value of V_{X} in mL (for the three MCRR exchange chambers used in the three test setups), and the flow rate Q in mL/s.

### Example 5: Release from a Flow Medium of Cyclosporine Emulsion to an External Medium

A nano-emulsion comprising cyclosporine, polysorbate 80, castor oil and a polymer was obtained, and a release test was performed following the same MCRR setup and steps listed in Example 4:
a) Immerse the MCRR probe exchange chamber sufficiently in a stirred aqueous external medium so exchange of said diffusible agent can occur between the flow medium and external medium;
b) Pump the flow medium through the probe at a constant Q;
c) Collect samples from the reservoir at times corresponding to selected exposure times, where the times were calculated from the exposure time and the test setup;
d) Repeat steps (b) and (c) until all samples are collected.
e) Perform an appropriate assay (HPLC, etc.) on each collected sample to determine the concentration and mass of ibuprofen in each sample;
f) Plot the percent of cyclosporine released vs. the time and the exposure time.

The release test was performed in triplicate, using three MCRR setups to perform one test each. Each setup comprised one MCRR probe, one reservoir, and one external medium. The nominal setups were the same for each setup and test (within experimental setup error). In each setup, reservoir and external medium were both continuously stirred.

The MCRR setup was as follows:
Q=0.040 mL/min (0.000667 mL/sec).
V_{T,1}=1.000 mL (average of the V_{T,l} for the three MCRR test setups)
V_{S}=0.090 mL/sample
V_{X}=0.007864 mL (average of V_{X} for the three exchange chambers used in the tests)
Flow medium: cyclosporine nano-emulsion
Initial flow medium cyclosporine concentration=483.5 μg/mL
External medium=1000 mL of NaCl in water (9 g/L)
Sample exposure times: 15, 30, 60, 120, 240, 300, 1260, 1320 seconds
Sample times: 31.8, 60.7, 112.9, 205.8, 368.7, 438.7, 1375.6, 1422.7 minutes (average of the calculated sample times corresponding to the sample exposure times, taken over the three MCRR test setups)

In this example, the flow medium is a nano-emulsion with oil globules dispersed in an aqueous continuous phase. Thus, in addition to release of the drug dissolved in the aqueous continuous phase of the emulsion, there is also an induced redistribution process in which the cyclosporine transfers out of the globules to replace the cyclosporine molecules lost from the aqueous phase due to the release. It is known that the transfer of a drug such as cyclosporine, which is much more soluble in oil than water, will be slow. Thus, the release and redistribution timeframes are anticipated to be significantly different.

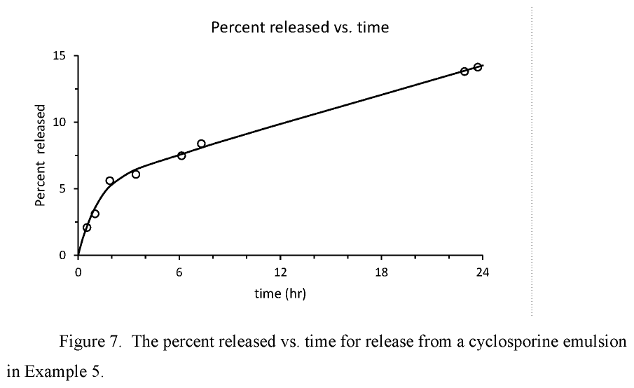

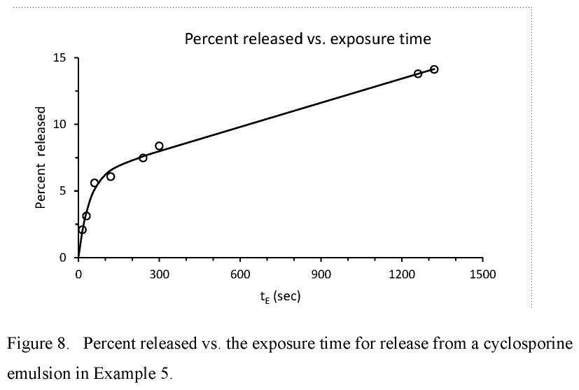

FIG. 7 shows the percent released vs. the time and FIG. 8 shows the percent released vs. the exposure time. In this example, less than 15% of the cyclosporine is released during the test, even though the exposure time went to 1320 seconds (vs. 300 seconds in Example 4) and the MCRR release test ran for about 24 hours.

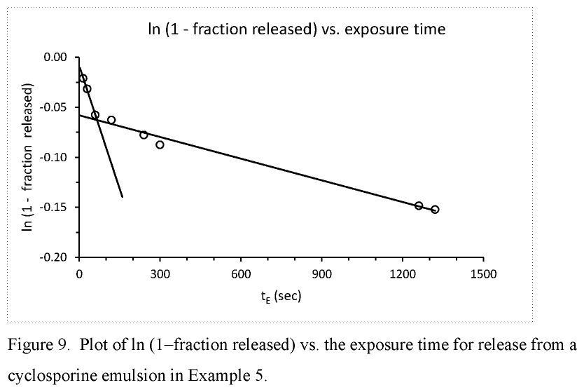

An important distinction between Example 4 and Example 5 is displayed in FIG. 9, which shows a plot of the log fraction remaining vs. the exposure time. Unlike the solution plot FIG. 6 from Example 4, which was linear, the analogous plot for release from cyclosporine nano-emulsion is not linear and shows two types of behavior, an approximately linear behavior at early times followed by another nearly linear phase but with a much less steep slope. The different slopes are also shown in FIGS. 7 and 8, which display a rapid initial release phase followed by a slower release phase. The initial rapid release phase corresponds to release of the drug initially in the aqueous continuous emulsion phase. The later, slower release phase corresponds to the slower transfer of the cyclosporine from the oil globules that becomes rate-limiting after most of the drug initially in the aqueous phase has been released (so the aqueous phase concentration becomes much lower than its initial value).

Without intending to be bound or limited by any model, it is possible to estimate the initial distribution of the cyclosporine by fitting the release data to the following empirical equation that has been found to represent the data well:

$$\% released = ( 1 - exp ( - {at}_{E} ) ) [ A + ( 100 - A ) ( 1 - exp ( - {bt}_{E} ) ) ] \qquad ( 11 )$$

where A represents the percent of the cyclosporine initially in the aqueous continuous emulsion phase, a is a rate constant that characterizes the timeframe of the release of drug initially in the aqueous phase, and b is a rate constant that characterizes the slower transfer and availability for release) from the oil globules to the aqueous phase. A fit of Eq. (11) to the data shown in FIG. 8 resulted in A=6.2%, indicating that 6.2% of the cyclosporine was initially dissolved in the aqueous phase of the nano-emulsion and 93.8% was initially in the globules. (This is consistent with known properties of cyclosporine, which is much more soluble in oil than water.)

### Example 6: Release from a Flow Medium Comprising a Drug Suspension to an External Medium

The flow medium comprises a drug with poor aqueous solubility that is less than about 10% dissolved in an aqueous medium. The release test is performed following the same MCRR setup, as illustrated by FIG. 1.

In this example, the reservoir is continously stirred. The external medium is also continuously stirred, and its volume is large compared to the total volume of the flow medium, so sink conditions are maintained.

The MCRR test is performed as follows:
a) Immerse the MCRR probe exchange chamber sufficiently in a stirred aqueous external medium so exchange of said diffusible agent can occur between the flow medium and external medium;
b) Pump the flow medium through the probe at a constant Q;
c) Collect samples from the reservoir at times corresponding to selected exposure times, where the times were calculated from the exposure time and the test setup;
d) Repeat steps (b) and (c) until all samples are collected.
e) Perform an appropriate assay (HPLC, etc.) on each collected sample to determine the concentration and mass of said drug in each sample;
f) Plot the percent of the released vs. the time and the exposure time.

The MCRR test setup is as follows: .

$$Q = 0 \text{.01} - 0.1 mL / min$$

$${V}_{T , 1} = 0.3 - 2. mL$$

$${V}_{S} = 0.02 - 0.1 mL / sample$$

$${V}_{X} = 1 - 10 μL$$

Flow medium: comprising drug or agent, as per product label
Initial flow medium drug concentration: per product label
External medium=appropriate medium, 20-1000 mL
Sample exposure times: 15, 30, 60, 120, 240, 300, 1260, 1320 seconds
Sample times: as determined from Eq. (4)

In this example, the flow medium comprises a suspension of a drug that is dispersed and partly dissolved in an aqueous continuous phase. Thus, in addition to release of the drug dissolved in the aqueous continuous phase of the suspension, there is also an induced redistribution process in which the drug transfers from the undissolved particles to replace the drug lost from the aqueous phase due to the release.

### Example 7: Release from a Flow Medium Comprising a Drug Complexed with a Protein to an External Medium

The flow medium comprises a drug with poor aqueous solubility that is complexed with an aqueous soluble protein such as human serum albumin, where less than 10% of the drug is in the free form and dissolved in an aqueous medium. The release test is performed following the same MCRR setup, as illustrated by FIG. 1.

In this example, the reservoir is continously stirred. The external medium is also continuously stirred, and its volume is large compared to the total volume of the flow medium, so sink conditions are maintained.

The MCRR test is performed as follows:
a) Immerse the MCRR probe exchange chamber sufficiently in a stirred aqueous external medium so exchange of said diffusible agent can occur between the flow medium and external medium;
b) Pump the flow medium through the probe at a constant Q;
c) Collect samples from the reservoir at times corresponding to selected exposure times, where the times were calculated from the exposure time and the test setup;
d) Repeat steps (b) and (c) until all samples are collected.
e) Perform an appropriate assay (HPLC, etc.) on each collected sample to determine the concentration and mass of said drug in each sample;
f) Plot the percent of the drug released vs. the time and the exposure time.

The MCRR test setup is as follows:

$$Q = 0 \text{.01} - 0.1 mL / min$$

$${V}_{T , 1} = 0 \text{.3} - 2. mL$$

$${V}_{S} = 0.02 - 0.1 mL \text{/sample}$$

$${V}_{X} = 1 - 10 μL$$

Flow medium: comprising drug or agent, as per product label
Initial flow medium total drug concentration (free and bound): per product label
External medium=appropriate medium, 20-1000 mL
Sample exposure times: 15, 30, 60, 120, 240, 300, 1260, 1320 seconds
Sample times: as determined from Eq. (4)
Exchange chamber membrane MWCO: not more than about 20 kD

In this example, the flow medium comprises a solution of a water-soluble protein, to which a drug with limited aqueous solubility is bound. The soluble protein molecules are dispersed in the aqueous phase. Most of the drug is bound to the soluble protein and a limited fraction of the drug is free and dissolved in the aqueous medium. Thus, in addition to release of the drug dissolved in the aqueous continuous phase of the suspension, there is also an induced redistribution process in which the drug transfers from the bound or complexed form to the free, dissolved form to replace the drug lost from the aqueous phase due to the release. Also, because of the molecular weight cutoff of the chosen exchange chamber membrane (less than about 20 kD), the protein will not be able to pass through the membrane (the molecular weight of human serum albumin is greater than 60 kD) and will remain in the flow medium.

### Example 8: Release from a Flow Medium Comprising a Drug Complexed with a Protein to an External Medium with Sample Volume Replacement: Replacement Fluid Comprises a Protein Solution

The flow medium comprises a drug with poor aqueous solubility that is complexed with am aqueous soluble protein such as human serum albumin, where less than 10% of the drug is in the free form and dissolved in an aqueous medium. The release test is performed following the same MCRR setup, as illustrated by FIG. 1.

In this example, the reservoir is continously stirred, and the volume withdorwan from the reservoir is replaced after sampling with a replacement fluid of the same composition as the initial flow medium but without any drug. The external medium is also continuously stirred, and its volume is large compared to the total volume of the flow medium, so sink conditions are maintained.

The MCRR test is performed as follows:
a) Immerse the MCRR probe exchange chamber sufficiently in a stirred aqueous external medium so exchange of said diffusible agent can occur between the flow medium and external medium;
b) Pump the flow medium through the probe at a constant Q;
c) Collect samples from the reservoir at times corresponding to selected exposure times, where the times were calculated from the exposure time and the test setup;
d) Immediately after withdrawing each sample, add replacement medium to said reservoir in a volume equal to the sample volume withdrawn;
e) Repeat steps (b), (c) and (d) until all samples are collected.
f) Perform an appropriate assay (HPLC, etc.) on each collected sample to determine the concentration and mass of said drug in each sample;
g) Plot the percent of the drug released vs. the time and the exposure time.

The MCRR test setup is as follows:

$$Q = 0 \text{.01} - 0.1 mL / min$$

$${V}_{T , 1} = 0 \text{.3} - 2. mL$$

$${V}_{S} = 0.02 - 0.1 mL \text{/sample}$$

$${V}_{X} = 1 - 10 μL$$

Flow medium: comprising a drug and water soluble protein in a drug-protein complex, as per product label
Replacement fluid: same composition as the flow medium except for containing no drug
Initial flow medium total drug concentration (free and bound): per product label
External medium=appropriate medium, 20-1000 mL
Sample exposure times: 15, 30, 60, 120, 240, 300, 1260, 1320 seconds
Sample times: as determined from Eq. (4)
Exchange chamber membrane MWCO: not more than about 20 kD

In this example, the flow medium comprises a solution of a water-soluble protein, to which a drug with limited aqueous solubility is bound. The soluble protein molecules are dispersed in the aqueous phase. Most of the drug is bound to the soluble protein and a limited fraction of the drug is free and dissolved in the aqueous medium. Thus, in addition to release of the drug dissolved in the aqueous continuous phase of the suspension, there is also an induced redistribution process in which the drug transfers from the bound or complexed form to the free, dissolved form to replace the drug lost from the aqueous phase due to the release. Also, because of the molecular weight cutoff of the exchange chamber membrane (less than about 20 kD), the protein will not be able to pass through the membrane (the molecular weight of human serum albumin is greater than 60 kD) and will remain in the flow medium.

Because of the volume replacement by the replacement fluid, there will be a dilution effect on the total drug concentration in the flow medium but not for the protein.

### Example 9: Release from a Flow Medium Comprising a Drug Complexed with a Protein to an External Medium with Sample Volume Replacement: Replacement Fluid is an Aqueous Buffer

The flow medium comprises a drug with poor aqueous solubility that is complexed with an aqueous-soluble protein such as human serum albumin, where less than 10% of the drug is in the free form and dissolved in an aqueous medium. The release test is performed following the same MCRR setup, as illustrated by FIG. 1.

In this example, the reservoir is continously stirred, and the volume withdorwan from the reservoir is replaced after sampling with an aqueous solution containing no drug or protein as the replacement fluid. The external medium is also continuously stirred, and its volume is large compared to the total volume of the flow medium, so sink conditions are maintained.

The MCRR test is performed as follows:
a) Immerse the MCRR probe exchange chamber sufficiently in a stirred aqueous external medium so exchange of said diffusible agent can occur between the flow medium and external medium;
b) Pump the flow medium through the probe at a constant Q;
c) Collect samples from the reservoir at times corresponding to selected exposure times, where the times were calculated from the exposure time and the test setup;
d) Immediately after withdrawing each sample, add replacement fluid to said reservoir in a volume equal to the sample volume withdrawn;
e) Repeat steps (b), (c) and (d) until all samples are collected.
f) Perform an appropriate assay (HPLC, etc.) on each collected sample to determine the concentration and mass of said drug in each sample;
g) Plot the percent of the drug released vs. the time and the exposure time.

In this example, the reservoir is continously stirred, and the volume withdorwan from the reservoir is replaced after sampling with a replacement fluid of the same composition as the initial flow medium but without any drug. The external medium is also continuously stirred, and its volume is large compared to the total volume of the flow medium, so sink conditions are maintained.

The MCRR test setup is as follows:

$$Q = 0 \text{.01} - 0.1 mL / min$$

$${V}_{T , 1} = 0 \text{.3} - 2. mL$$

$${V}_{S} = 0.02 - 0.1 mL \text{/sample}$$

$${V}_{X} = 1 - 10 μL$$

Flow medium: comprising a drug and water soluble protein in a drug-protein complex, as per product label
Replacement fluid: 25 mM of pH 7 phosphate buffer solution containing no drug or protein
Initial flow medium total drug concentration (free and bound): per product label
External medium=appropriate medium, 20-1000 mL
Sample exposure times: 15, 30, 60, 120, 240, 300, 1260, 1320 seconds
Sample times: as determined from Eq. (4)

In this example, the flow medium comprises a solution of a water-soluble protein, to which a drug with limited aqueous solubility is bound. The soluble protein molecules are dispersed in the aqueous phase. Most of the drug is bound to the soluble protein and a limited fraction of the drug is free and dissolved in the aqueous medium. Thus, in addition to release of the drug dissolved in the aqueous continuous phase of the suspension, there is also an induced redistribution process in which the drug transfers from the bound or complexed form to the free, dissolved form to replace the drug lost from the aqueous phase due to the release.

Also, because of the molecular weight cutoff of the exchange chamber membrane (less than about 20 kD), the protein will not be able to pass through the membrane (the molecular weight of human serum albumin is greater than 60 kD) and will remain in the flow medium.

Because of the volume replacement by the replacement fluid, there will be a dilution effect on the both the total drug concentration and the protein concentration in the flow medium.

### Example 10: Simultaneous Release from a Flow Medium to Two External Media and Uptake by the Flow Medium from One External Medium Using Two MCRR probes in Parallel

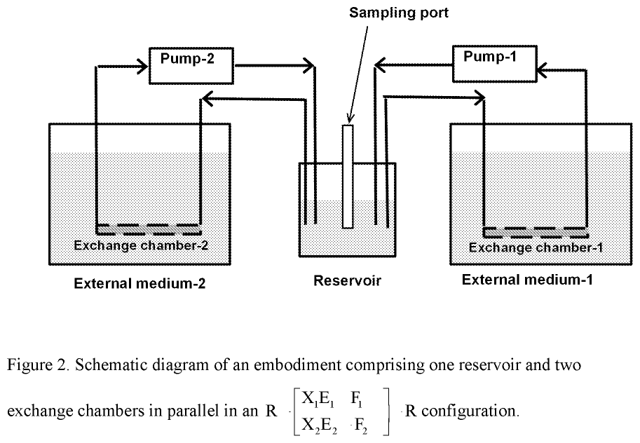

The release test is performed following the MCRR setup illustrated by FIG. 2. Two MCRR probes (probe-1 and probe-2) are used and share the same reservoir and flow medium in said reservoir, but each exchange chamber (X₁ and X₂) is immersed in a separate external medium (E₁ and E₂). The volumes of each exchange chamber are denoted by V_{X1} and V_{X2}. External medium E₁ comprises only an aqueous buffer and external medium E₂ comprises an aqueous buffer and another agent that exchanges between E₂ and the flow medium. The flow medium initially contains the drug but not the other agent, releases the drug to both E₁ and E₂, and accumulates the agent from E₂.

The MCRR test is performed as follows:
a) Immerse each MCRR probe exchange chamber into its stirred external medium (X₁ in E₁ and X₂ in E₂) sufficiently so exchange of said diffusible agents can occur between the flow medium and each external medium;
b) Pump the flow medium through probe-1 at a constant rate Q₁ and through probe-2 at a constant rate Q₂;
c) Collect samples from the reservoir at predetermined times, calculating the exposure time for the drug and the agent separately from the sampling times and the MCRR test setup;
d) Repeat steps (b) and (c) until all samples are collected.
e) Perform an appropriate assay (HPLC, etc.) on each collected sample to determine the concentration and mass of said drug and agent in each sample;
f) Plot the mass or percent of the drug released vs. the time and the exposure time, and plot the mass of agent taken up by the flow medium vs. the time and exposure time.

In this example, the reservoir is continously stirred. The external media E₁ and E₂ are both continuously stirred, and the volume of each is large compared to the total volume of the flow medium, so sink conditions are maintained relative to the drug.

The MCRR test setup is as follows: .

$${Q}_{1} = 0 \text{.01} - 0.1 mL / min ; {Q}_{2} = 0.01 - 0.1 mL / min$$

V_{T,1}=1.0 mL
V_{S}=0.05 mL/sample
V_{X1}=5.0 μL; V_{X2}=10 μL;
Flow medium: aqueous solution initially comprising a drug but not said agent
Initial flow medium drug concentration: per product label
External medium E₁=20-1000 mL, containing no drug or agent
External medium E₂=20-1000 mL, containing agent but no drug
Sample times: 10, 30, 60, 120, 240, 480, 720, 1440 minutes, (600, 1800, 3600, 7200, 14400, 28800, 86400 seconds)
Exposure times for the drug: determined from the sample times, substituting the sum V_{X1}+V_{X2} for the term V_{X} in Eq. (5)
Exposure times for the agent: determined from the sample times, substituting V_{X2} for the term V_{X} in Eq. (5)

In this example, the flow medium comprises an aqueous drug solution that contains no agent. The flow medium releases the drug from both exchange chambers to both external media, so the total exchange chamber volume affecting the drug release is V_{X1}+V_{X2}. The flow medium also takes up the agent but only via X₂ from E₂, so the exchange chamber volume affecting the agent uptake is V_{X2}.

Since the sampling is done from the common reservoir, the sampling times and intervals are the same for the drug and the agent, but their exposure times are different. Table 3 shows the sampling times and how the corresponding exposure times are calculated for the drug and agent.

TABLE 3
Example of determining exposure times from sampling
times from Eq. (5) for the drug and agent.

| | | | | Exposure time interval | | Exposure time t_{E, j} | |
|---|---|---|---|---|---|---|---|
| Sampling | Time tⱼ | Δtⱼ | V_{T, j} | Δt_{E, j} (sec) | | (sec) | |
| Interval j | (sec) | (sec) | (μL) | Drug | Agent | Drug | Agent |
| 1 | 600 | 600 | 1000 | 9.0 | 2857 | 6.0 | 6.0 |
| 2 | 1800 | 1200 | 950 | 18.9 | 2600 | 12.6 | 18.6 |
| 3 | 3600 | 1800 | 900 | 30.0 | 2343 | 20.0 | 38.6 |
| 4 | 7200 | 3600 | 850 | 63.5 | 6257 | 42.4 | 81.0 |
| 5 | 14400 | 7200 | 800 | 135.0 | 5486 | 90.0 | 171.0 |
| 6 | 28800 | 14400 | 750 | 288.0 | 9429 | 192.0 | 363.0 |
| 7 | 43200 | 14400 | 700 | 308.6 | 19714 | 205.7 | 568.7 |
| 8 | 86400 | 43200 | 650 | 996.9 | 15857 | 664.6 | 1233.3 |

### Example 11: Release of a Drug from a Flow Medium Solution to an External Medium by a Reversing Flow Between Two Reservoirs

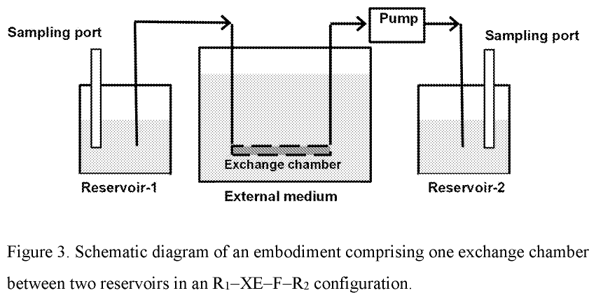

The release test is performed following the MCRR setup R₁-EX-F-R₂, as illustrated by FIG. 3. One MCRR probe is used with two reservoirs (R₁ and R₂) and the flow medium alternately is caused to flow from R₁ to R₂ at a known flow rate, then the movement is reversed and the flow medium is caused to flow from R₂ to R₁. The flow medium comprises a drug solution and the external medium initially contains no drug.

The MCRR test is performed as follows:
a) Immerse the MCRR probe exchange chamber sufficiently in a stirred aqueous external medium so exchange of the drug can occur between the flow medium and external medium;
b) Pump the flow medium through the MCRR probe at a constant Q until a preselected volume is transferred from R₁ to R₂;
c) Reverse the pumping direction to pump the flow medium through the MCRR probe at a constant Q until another preselected volume is transferred from R₂ to R₁;
d) Collect samples from one or both reservoirs at times corresponding to the selected exposure times, where the times are calculated from the exposure time and the test setup;
e) Repeat steps (b), (c) and (d) until all samples are collected;
f) Perform an appropriate assay (HPLC, etc.) on each collected sample to determine the concentration and mass of ibuprofen in each sample;
g) Plot the percent of the drug released vs. the time and the exposure time.

In this example, the reservoir is continuously stirred. The external medium is also continuously stirred, and its volume is large compared to the total volume of the flow medium, so sink conditions are maintained relative to the drug.

The MCRR test setup is as follows:

$$Q = 0 \text{.01} - 0.1 mL / min ( in both directions )$$

$${V}_{T , 1} = 0 \text{.5} - 5 mL$$

$${V}_{S} = 0.05 mL \text{/sample}$$

$${V}_{X} = 0.001 - 0.01 mL$$

Flow medium: drug solution
Initial flow medium drug concentration=determined from product label
External medium=1000 mL of 25 mM phosphate buffer pH=7 at 20° C.
Sample exposure times: 15, 30, 60, 120, 180, 300 seconds
Sample times: determined from the MCRR setup and Eq. (4)

In this MCRR release test setup, there is only one exchange chamber, and the exposure time and sampling times are related by Eq. 4. Sampling can be done from R₁ or R₂, both simultaneously or both at different times.

### Example 12. Description of an Exchange Chamber Comprising Two Different Exchange Chamber Membranes

An example is given herein to illustrate embodiments of MCRR comprising two exchange chambers X₁ and X₂ that are connected by impermeable tubing and immersed in the same external medium, and which withdraw the flow medium from and return it to a single reservoir. This is described by the notation R-(X₁-X₂)E-F-R, where the notation -(X₁-X₂)E- denotes two exchange chambers immersed in the same external medium such that the flow medium flows through them in series.

In a preferred embodiment, the length of impermeable tubing that separates X₁ and X₂ is less than about 5 cm, preferably less than about 2 cm, and more preferably less than about 1 cm. The exchange chambers can have different lengths and membrane properties. (The parameters detailed in the embodiments that follow are typical and serve as examples but are not meant to limit any ranges that fall within the scope of the invention.)

In a preferred embodiment, both exchange chambers X₁ and X₂ comprise a commercially available tubular segment of permeable regenerated cellulose membrane with a MWCO of 13 kD and a nominal inner radius a of about 0.01-0.05 cm (for instance, Spectra/Por Hollow Fiber membranes, Repligen, Waltham MA). The length of each exchange chamber X₁ and X₂ is about 1-15 cm and the volume V_{X} ranges from less than 1 μL to more than 10 μL for each, but the length and volume of X₁ and X₂ do not have to be the same.

In another preferred embodiment, exchange chamber X₁ comprises a commercially available tubular segment of permeable regenerated cellulose membrane with a MWCO of 13 kD and a nominal inner radius a of about 0.01-0.05 cm. Exchange chamber X₂ comprises a tubular segment of a different permeable membrane with a MWCO of about 1 kD to about 10 kD, a nominal inner radius a of about 0.01-0.05 cm, and a length of about 1-15 cm.

In yet another preferred embodiment, exchange chamber X₁ comprises a commercially available tubular segment of permeable regenerated cellulose membrane with a MWCO of 13 kD and a nominal inner radius a of about 0.01-0.05 cm. Exchange chamber X₂ comprises a tubular segment of a different permeable membrane with a MWCO of about 20 kD to more than about 2 MDa (million daltons), a nominal inner radius a of about 0.01-0.1 cm, and a length of about 1-15 cm.

In still another preferred embodiment, exchange chamber X₁ comprises a commercially available tubular segment of permeable regenerated cellulose membrane with a MWCO of 13 kD and a nominal inner radius a of about 0.01-0.05 cm. The other exchange chamber X₂ comprises a different, “nonlinear” geometry (herein, “nonlinear geometry” refers to a geometry that is not a simple connection of straight cylindrical tubing to form a cylindrical geometry) with a MWCO from about 6 kD to about 2 MDa. (For instance, CMA 7, CMA 8, CMA 11, CMA 12, etc. Harvard Apparatus, Holliston, MA)

In even another preferred embodiment, both exchange chamber X₁ and exchange chamber X₂ comprise a nonlinear geometry, in which X₁ and X₂ may be the same or different, and with the MWCO of both X₁ and X₂ being about 2 kD to about 2 MDa.

In the above and other embodiments, the exchange chamber described above may be in arranged in the reverse order, so the flow medium flows through X₂ then X₁, as denoted by R-(X₂-X₁)E-F-R. The configuration may also be modified by moving the location of the pump to form configuration R-F-(X₂-X₁)E-R.

The reservoir is in a 1-10 mL glass vessel that is sealed and has a sampling port through which a needle can be passed, and the volume of the external medium is typically 250-1000 mL. Samples of volume V_{S} are taken from the flow medium in the reservoir through the sample port using an accurate needle/syringe set. The reservoir is continuously stirred, and the flow through the MCRR probe is typically maintained at a constant flow rate Q in one direction.

The external medium is also continuously stirred, and its volume is typically from about 10-1000 mL, so it is much greater than V_{X} and preferably much greater than the total volume of the flow medium VT, so sink conditions are maintained in the external medium.

This configuration can be used for release tests (the flow medium comprises a formulation composition with a drug that is at least partially dissolved and the external medium is initially void of the drug, so the drug is released from the flow medium to the external medium), or for uptake tests (the external medium comprises a formulation composition with a drug that is at least partially dissolved and the flow medium is initially void of the drug, so the drug is taken up by the flow medium from the external medium).

### REFERENCES

Bellantone RA (2012). U.S. Pat. No. 8,333,107.
Kabir MA, Taft DR, Joseph CK, Bellantone RA. Measuring drug concentrations using pulsatile microdialysis: Theory and method development in vitro. Int. J. Pharmaceutics 293 (2005), 171-182.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method for determining the quantity of a diffusible agent that is exchanged between two media, comprising:
a) providing at least one exchange chamber comprising a section of relatively highly permeable membrane through which a diffusible agent is to be transferred, and attached to and positioned between an inlet to a reservoir or source of flow medium and an outlet to said reservoir, where the inlet and outlet materials are relatively impermeable to said diffusible agent;
b) putting said exchange chamber in contact with an external medium by immersing it sufficiently in the external medium so exchange of said diffusible agent can occur between the flow medium and external medium;
c) perfusing a known quantity of a flow medium through said relatively impermeable tubing and said exchange chamber, and back to said reservoir at a controlled flow rate;
d) at a known time, withdrawing a sample of known volume of said flow medium from said reservoir, and optionally replacing said withdrawn sample by adding an equal volume of a replacement liquid of known composition to said reservoir;
e) determining the mass of said diffusible agent in said flow medium sample, and determining the mass exchanged between said flow medium and said external medium during step (c);
f) optionally continuing step (c) and repeating steps (d) and (e) for a selected time or number of samples.

2. A method of claim 1 in which:
a) the volume of each sample withdrawn from the reservoir is not replaced with a replacement fluid;
b) the initial total volume of the flow medium V_{T}(l) and the volume of the exchange chamber V_{X} are known, and the exposure times t_{E,j} are preselected;
c) the times after the start of a release test at which samples are withdrawn from the reservoir tⱼ are calculated from Eq. (4).

3. A method of claim 1 in which:
a) the volume of each sample withdrawn from the reservoir is replaced with an equal volume of replacement fluid of known composition;
b) the total volume of the flow medium V_{T}(l) and the volume of the exchange chamber V_{X} are known, and the exposure times t_{E,j} are preselected;
c) the times after the start of a release test at which samples are withdrawn from the reservoir tⱼ are calculated from Eq. (7).

4. A method of claim 1 in which the flow medium comprises a drug composition in which the drug is at least partially dissolved, and the external medium is a liquid initially containing substantially none of, or a known amount of, said drug.

5. (canceled)

6. (canceled)

7. A method of claim 1 in which the flow medium comprises a drug solution or an emulsion containing a drug or a suspension containing a drug and transfers or releases at least some drug to the external medium.

8. A method of claim 1 in which the flow medium is an aqueous medium comprising a drug complexed or bound to a carrier which is a protein or polymer and transfers at least some drug to the external medium.

9. A method of claim 1 in which the external medium comprises an agent that chemically reacts with one or more drugs or agents transferred from the flow medium to said external medium.

10. A method of claim 1 in which the external medium comprises a drug composition in which the drug is at least partially dissolved. and flow medium is initially void of the drug and at least some drug transfers to the flow medium.

11. (canceled)

12. (canceled)

13. A method of claim 1 in which the flow medium comprises an aqueous solution or an emulsion or a suspension such that said aqueous solution or emulsion or suspension initially contain substantially none of, or a known amount of, said drug and at least some drug transfers to the flow medium.

14. A method of claim 1 in which the flow medium comprises an aqueous solution or dispersion of a carrier which is a protein or polymer such that said flow medium initially contains substantially none of, or a known amount of, said drug and at least some drug transfers to the flow medium.

15. A method of claim 1 in which the flow medium comprises an aqueous medium comprising an agent that chemically reacts with one or more drugs or agents transferred to the flow medium from the external medium.

16. A method of claim 1 in which the volume of the reservoir is zero and the inlet and outlet impermeable tubing are connected as a closed loop, so all of the flow medium is contained in the impermeable tubing and exchange chamber.

17. A method of claim 1 in which there are multiple exchange chambers that may comprise different properties and optionally multiple external media that may comprise different properties.

18. (canceled)

19. A method of claim 1 in which there are multiple exchange chambers connected in in series or in parallel or a combination thereof that may comprise different properties and optionally multiple external media that may comprise different properties.

20. (canceled)

21. A method of claim 1 in which the flow rate is held constant, varied in a known manner, or both.

22. A method of claim 1 in which the properties of the external medium which are its composition, temperature, pH, and stirring speed are caused to change in a known manner with time.

23. A method of claim 1 in which the properties of the reservoir which are its composition, temperature, pH, or stirring speed are caused to change in a known manner with time.

24. A method of claim 1 in which the flow is continuous for some time duration, then stopped to reverse the flow direction.

25. A method for determining the quantity of a diffusible agent that is exchanged between two media, comprising:
a) providing a first exchange chamber comprising a section of relatively highly permeable membrane through which a diffusible agent is to be transferred and attached to and positioned between an inlet to a single, common reservoir or source of flow medium and an outlet to said reservoir, where the inlet and outlet materials are relatively impermeable to said diffusible agent;
b) providing a second exchange chamber comprising a section of relatively highly permeable membrane through which a diffusible agent is to be transferred and attached to and positioned between an inlet to said common reservoir or source of flow medium and an outlet to said reservoir, where the inlet and outlet materials are relatively impermeable to said diffusible agent;
c) putting said first exchange chamber in contact with a first external medium by immersing it sufficiently in said first external medium so exchange of said diffusible agent can occur between said flow medium and said first external medium;
d) putting said second exchange chamber in contact with a second external medium by immersing it sufficiently in said first external medium so exchange of said diffusible agent can occur between said flow medium and said second external medium;
e) simultaneously perfusing a known quantity of flow medium from said common reservoir, through said relatively impermeable tubing and said first exchange chamber, and back to said common reservoir at a controlled flow rate, and perfusing a known quantity of flow medium from said common reservoir, through said relatively impermeable tubing and said second exchange chamber, and back to said common reservoir at a controlled flow rate;
f) at a known time, withdrawing a sample of known volume of said flow medium from said common reservoir, and optionally replacing said withdrawn sample by adding an equal volume of a replacement liquid of known composition to said common reservoir;
g) determining the mass of said diffusible agent in said flow medium sample, and determining the mass exchanged between said flow medium and said first external medium and said second external medium during step (e);
h) optionally continuing step (e) and repeating steps (f) and (g) for a selected time or number of samples.

26. A method of claim 25 in which:
a) the flow medium comprises a drug composition in which the drug is at least partially dissolved;
b) a first external medium that is a liquid initially containing substantially none of, or a known amount of, said drug;
c) a second external medium is a liquid initially containing substantially none of, or a known amount of, said drug but contains a diffusible agent, such that said flow medium and said first external medium initially contain substantially none of, or a known amount of, said agent;
d) the volume of each sample withdrawn from the reservoir is not replaced with a replacement fluid;
e) the initial total volume of the flow medium V_{T}(l) and the volume of the first exchange chamber V_{X1} and the second exchange chamber V_{X2} are known, and the sample times t are preselected;
f) the exposure times t_{E,j} for said drug are calculated from the times at which samples are withdrawn from the reservoir tⱼ by Eq. (4) using the sum of the volumes V_{X1}+V_{X2} for the term V_{X};
g) the exposure times t_{E,j} for the agent are calculated from the times at which samples are withdrawn from the reservoir tⱼ by Eq. (4) using the volume V_{X2} for the term V_{X}.

27. A method of claim 25 in which:
a) the flow medium comprises a drug composition in which the drug is at least partially dissolved;
b) a first external medium that is a liquid initially containing substantially none of, or a known amount of, said drug;
c) a second external medium is a liquid initially containing substantially none of, or a known amount of, said drug but contains a diffusible agent, such that said flow medium and said first external medium initially contain substantially none of, or a known amount of, said agent;
d) the volume of each sample withdrawn from the reservoir is replaced with an equal volume of a replacement fluid of known composition;
e) the initial total volume of the flow medium V_{T}(l) and the volume of the first exchange chamber V_{X1} and the second exchange chamber V_{X2} are known, and the sample times tⱼ are preselected;
f) the exposure times t_{E,j} for said drug are calculated from the times at which samples are withdrawn from the reservoir tⱼ by Eq. (7) using the sum of the volumes V_{X1}+V_{X2} for the term V_{X};
g) the exposure times t_{E,j} for the agent are calculated from the times at which samples are withdrawn from the reservoir tⱼ by Eq. (7) using the volume V_{X2} for the term V_{X}.

28. A method of claim 25 in which the flow medium comprises a drug composition in which the drug is at least partially dissolved, and the external medium is a liquid initially containing substantially none of, or a known amount of, said drug.

29. (canceled)

30. (canceled)

31. A method of claim 25 in which the flow medium comprises a drug solution or an emulsion containing a drug or a suspension containing a drug and transfers or releases at least some drug to the external medium.

32. A method of claim 25 in which the flow medium is an aqueous medium comprising a drug complexed or bound which is a protein or polymer and transfers at least some drug to the external medium.

33. A method of claim 25 in which the external medium comprises an agent that chemically reacts with one or more drugs or agents transferred from the flow medium to said external medium.

34. A method of claim 25 in which the external medium comprises a drug composition in which the drug is at least partially dissolved. and flow medium is initially void of the drug and at least some drug transfers to the flow medium.

35. (canceled)

36. (canceled)

37. A method of claim 25 in which the flow medium comprises an aqueous solution or an emulsion or a suspension such that said aqueous solution or emulsion or suspension initially contain substantially none of, or a known amount of, said drug and at least some drug transfers to the flow medium.

38. A method of claim 25 in which the flow medium comprises an aqueous solution or dispersion of a carrier which is a protein or polymer such that said flow medium initially contains substantially none of, or a known amount of, said drug and at least some drug transfers to the flow medium.

39. A method of claim 25 in which the flow medium comprises an aqueous medium comprising an agent that chemically reacts with one or more drugs or agents transferred to the flow medium from the external medium.

40. A method of claim 25 in which there are multiple exchange chambers that may comprise different properties and optionally multiple external media that may comprise different properties.

41. (canceled)

42. A method of claim 25 in which there are multiple exchange chambers connected in series or in parallel or a combination thereof that may comprise different properties and optionally multiple external media that may comprise different properties.

43. (canceled)

44. A method of claim 25 in which the flow rate is held constant, varied in a known manner, or both.

45. A method of claim 25 in which the properties of the external medium which are its composition, temperature, pH, or stirring speed are caused to change in a known manner with time.

46. A method of claim 25 in which the properties of the reservoir which are its composition, temperature, pH, pr stirring speed are caused to change in a known manner with time.

47. A method of claim 25 in which the flow is continuous for some time duration, then stopped to reverse the flow direction.

48. A method for determining the quantity of a diffusible agent that is exchanged between two media, comprising:
a) providing at least one exchange chamber comprising a section of relatively highly permeable membrane through which a diffusible agent is to be transferred, and attached and positioned between a first segment of tubing that can accept flow medium from or supply it to a first reservoir, and a second segment of tubing that can accept flow medium from or supply it to a second reservoir, where said first and said second segments of tubing are relatively impermeable to said diffusible agent;
b) putting said exchange chamber in contact with an external medium by immersing it sufficiently in the external medium so exchange of said diffusible agent can occur between the flow medium and external medium;
c) perfusing a known quantity of a flow medium from the first reservoir, through said relatively impermeable tubing and said exchange chamber, and to the second reservoir at a controlled flow rate;
d) at a known time, withdrawing a sample of flow medium from either reservoir, determining the mass of said diffusible agent in said flow medium sample, and optionally replacing said withdrawn sample by adding an equal volume of a replacement liquid of known composition to said reservoir from which said sample was taken;
e) determining the mass of said diffusible agent in said flow medium sample, and determining the mass exchanged between said flow medium and said external medium during step (c);
f) optionally reversing the flow direction of the flow medium, perfusing said flow medium from the second reservoir, through impermeable tubing and said exchange chamber, and to said first reservoir at a controlled flow rate that may be the same or different from the flow rate in step (c);
g) at a known time, withdrawing a sample of flow medium from either reservoir, determining the mass of said diffusible agent in said flow medium sample, and optionally replacing said withdrawn sample by adding an equal volume of a replacement liquid of known composition to said reservoir from which said sample was taken;
h) optionally, repeating steps (c) through (f) using the same or different flow rates, for a selected time or number of samples.

49. A method of claim 48 in which:
a) the volume of each sample withdrawn from the reservoir is not replaced with a replacement fluid;
b) the initial total volume of the flow medium V_{T}(l) and the volume of the exchange chamber V_{X} are known, and the exposure times t_{E,j} are preselected;
c) the times after the start of a release test at which samples are withdrawn from the reservoir tⱼ are calculated from Eq. (4).

50. A method of claim 48 in which:
a) the volume of each sample withdrawn from the reservoir is replaced with an equal volume of a replacement fluid of known composition;
b) the total volume of the flow medium V_{T}(l) and the volume of the exchange chamber V_{X} are known, and the exposure times t_{E,j} are preselected;
c) the times after the start of a release test at which samples are withdrawn from the reservoir tⱼ are calculated from Eq. (7).

51. A method of claim 48 in which the flow medium comprises a drug composition in which the drug is at least partially dissolved, and the external medium is a liquid initially containing substantially none of, or a known amount of, said drug.

52. (canceled)

53. (canceled)

54. A method of claim 48 in which the flow medium comprises a drug solution or an emulsion containing a drug or a suspension containing a drug and transfers or releases at least some drug to the external medium.

55. A method of claim 48 in which the flow medium is an aqueous medium comprising a drug complexed or bound to a carrier which is a protein or polymer and transfers at least some drug to the external medium.

56. A method of claim 48 in which the external medium comprises an agent that chemically reacts with one or more drugs or agents transferred from the flow medium to said external medium.

57. A method of claim 48 in which the external medium comprises a drug composition in which the drug is at least partially dissolved. and flow medium is initially void of the drug and at least some drug transfers to the flow medium.

58. (canceled)

59. (canceled)

60. A method of claim 48 in which the flow medium comprises an aqueous solution or an emulsion or a suspension such that said aqueous solution or emulsion or suspension initially contain substantially none of, or a known amount of, said drug and at least some drug transfers to the flow medium.

61. A method of claim 48 in which the flow medium comprises an aqueous solution or dispersion of a carrier which is a protein or polymer such that said flow medium initially containing substantially none of, or a known amount of, said drug and at least some drug transfers to the flow medium.

62. A method of claim 48 in which the flow medium comprises an aqueous medium comprising an agent that chemically reacts with one or more drugs or agents transferred to the flow medium from the external medium.

63. A method of claim 48 in which the volume of the reservoir is zero and the inlet and outlet impermeable tubing are connected as a closed loop, so all of the flow medium is contained in the impermeable tubing and exchange chamber.

64. A method of claim 48 in which there are multiple exchange chambers that may comprise different properties and optionally multiple external media that may comprise different properties.

65. (canceled)

66. A method of claim 48 in which there are multiple exchange chambers connected in series or in parallel or a combination thereof that may comprise different properties and optionally multiple external media that may comprise different properties.

67. (canceled)

68. A method of claim 48 in which the flow rate is held constant, varied in a known manner, or both.

69. A method of claim 48 in which the properties of the external medium which are its composition, temperature, pH, or stirring speed are caused to change in a known manner with time.

70. A method of claim 48 in which the properties of the reservoir which are its composition, temperature, pH, or stirring speed are caused to change in a known manner with time.

71. A method of claim 48 in which the flow is continuous for some time duration, then stopped to reverse the flow direction.

72. (canceled)

73. (canceled)

74. (canceled)

75. (canceled)

76. (canceled)

77. An apparatus comprising a first holding means for holding a liquid containing as a component at least one poorly-soluble material, said first holding means communicating with at least one second holding means for holding a liquid received from said first holding means and in said second holding means being immersed a separation means for separating at least one of the components of said liquid, said second holding means communicating with said first holding means in a manner by which to return said liquid to said first holding means in a controllable recycle manner, and a powered transfer means to controllably move said liquid forward through said separation means and backward for said recycle, and any of said holding means optionally containing a sampling means for withdrawing samples from said liquid in said holding means.

78. An apparatus of claim 77 comprising an embodiment of FIG. 1, or FIG. 2, or FIG. 3.

79. (canceled)

80. (canceled)
